(19) United States
(12) Patent Application Publication
(10) Pub. No.: US 2017/0239346 A1
(43) Pub. Date: Aug. 24, 2017

(54) NOVEL COCAINE HAPTENS AND NANOFIBER-BASED COCAINE VACCINES

(71) Applicants: Jia Zhou, League City, TX (US); Jai S. Rudra, Galveston, TX (US); Kathryn A. Cunningham, Galveston, TX (US)

(72) Inventors: Jia Zhou, League City, TX (US); Jai S. Rudra, Galveston, TX (US); Kathryn A. Cunningham, Galveston, TX (US)

(21) Appl. No.: 15/441,474

(22) Filed: Feb. 24, 2017

Related U.S. Application Data
(60) Provisional application No. 62/299,225, filed on Feb. 24, 2016.
(60) Provisional application No. 62/299,840, filed on Feb. 25, 2016.

(51) Int. Cl. A61K 39/385; A61K 39/00

(52) U.S. Cl. CPC A61K 39/385; A61K 39/0013; A61K 2039/6012; A61K 2039/6031; A61K 2039/627

(57) ABSTRACT

Certain embodiments are directed to chemically defined self-adjuvanting cocaine vaccines composed of novel cocaine haptens and self-assembling peptide domains.

### PRIORITY PARAGRAPH

This application claims priority to U.S. Application No. 62/299,225 filed Feb. 24, 2016 and U.S. Application No. 62/299,840 filed Feb. 25, 2016, each of which is incorporated herein by reference in its entirety.

### STATEMENT REGARDING FEDERALLY SPONSORED RESEARCH AND DEVELOPMENT

This invention was made with government support under Grant No. R21 DA036663, awarded by the U.S. National Institute of Health and the National Institute on Drug Abuse. The government has certain rights in the invention.

### BACKGROUND

Cocaine use disorder remains one of the greatest challenges on the public health agenda in the USA with ca. over 900,000 Americans meeting the criteria for diagnosis in 2014 (Center for Behavioral Health Statistics and Quality, 2015). Despite the continued impact of this chronic health disorder, there are no FDA-approved therapeutics for either cocaine use disorder or for the management of acute overdose (Shen et al., 2012; Orson et al., 2014).

An emerging idea at the forefront of combating cocaine use disorder and forestalling overdose/toxicity is the development of vaccines, which generate cocaine-specific antibodies and prevent drug penetration across the blood-brain barrier (Orson et al., 2014). A number of anti-cocaine vaccines are at various stages of preclinical and clinical development (Orson et al., 2014; Kosten et al., Brit J Clin Pharmacol, 2014). However, a major limiting factor in the success of anti-cocaine vaccines is the degree and specificity of immunity evoked by the addictive drug analog (Moreno et al., 2011; Ramakrishnan et al., 2014). Further administration of small molecules like cocaine will not elicit an immune response unless it is covalently attached to an immunogenic carrier protein and co-administered with an adjuvant (Alving et al., 2014).

The current landscape of cocaine vaccine development relies heavily on developing antigenicity based upon the conjugation of the drug to carrier proteins from animal and bacterial sources and formulating the cocaine-carrier conjugates into emulsions using exogenous adjuvants (Alving et al., 2014). The positioning of chemical linkers on cocaine for coupling to carrier proteins has been shown to be crucial for proper immune stimulation (Ino et al., 2007). Cocaine haptens modified at the methyl ester (P1), benzoate (P2), N-methyl amino (P3) and two-carbon bridge (P4) moieties of cocaine have been previously reported (Moreno et al., 2009). However, most of these haptens did not reach clinical trials with the exception of a cholera toxin B-cocaine conjugate (TA-CD) vaccine having a linker at the P3 position (Ramakrishnan et al. 2014).

There is a severe lack of clinically approved adjuvants that are both effective and safe and in the United States. Aluminum-based salts, collectively called ‘alum’, or alum in combination with monophosphoryl lipid A (MPLA) are restricted only to use via intramuscular route of administration (Alving et al., 2014). Further, most experimental adjuvants under development are heterogeneous mixtures of plant or microbial extracts, which suffer from poor chemical definition and toxicity (Aguilar et al., 2007).

The TA-CD vaccine in combination with alum adjuvant has undergone multisite Phase III testing (Kosten, Drug and alcohol dependence, 2014). After 5 rounds of immunizations ˜33% of the patients failed to achieve anti-cocaine antibody titers but all patients had antibodies against cholera toxin (Kosten, Drug and alcohol dependence, 2014). Adverse events such as induration and erythema at the injection site were reported due to use of alum and further, alum is restricted to the intramuscular route, making it impossible to investigate needle-free avenues of vaccination that provide higher patient compliance rates (Aguilar et al., 2007; Kosten, Drug and alcohol dependence, 2014). Also chronic cocaine exposure leads to the development of anti-cocaine IgM antibodies and the presence of IgM antibodies has been shown to be a poor marker for eliciting IgG antibody responses to cocaine vaccines (Orson et al., 2013). Furthermore, cocaine exposure is associated with immune system suppression that could potentially lead to vaccine failures (Pellegrino et al., 2001). Therefore, there is a need for novel cocaine vaccine strategies that can overcome the immunosuppressive effects associated with chronic cocaine exposure compared to toxin-based carriers and alum adjuvants and yet can be safely administered without significant concerns for toxicity.

### SUMMARY

There is a clinical need for therapeutics for cocaine use disorder and for the management of acute overdose. In certain aspects, compositions and methods described herein can be used to treat and prevent cocaine use disorder and manage acute cocaine overdose.

Certain embodiments are directed to immunogenic compositions comprising a peptide fibril coupled to a plurality of cocaine haptens or antigens. A hapten as used herein is a small molecule that elicit an immune response only when attached to a carrier. A “cocaine hapten” or “cocaine antigen” refers to a molecule that induces antibodies that bind cocaine. In certain aspects the cocaine hapten or antigen comprises a compound of Formula I:

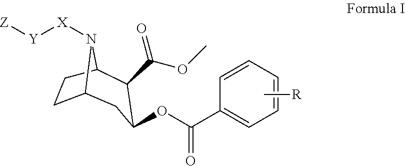

wherein X is —CH₂—, —CO—, or —CS—; Y is a substituted alkane, alkene, alkyne, or aromatic rings; Z is —CO₂H—, —OH, N₃, —NH₂, —CHO, —SH, —CH═CH₂, —C≡CH, or

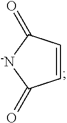

and R is hydrogen, hydroxyl, nitro, mercapto, cyano, azido, alkyl, heteroalkyl, alkoxy, halogen, amino, oxo, or alkylsulfonyl. In certain aspects R is in particular —F, —Cl, or —Br. In certain aspects R is at the 2, 3, 4, 5, or 6 position.

In certain aspects, the peptide fibril comprises a plurality of self-assembling peptides. The peptide fibril can have a length of at least 0.25, 0.5, 1, 10, 50 to 10, 25, 50, 100 μm, including all values and ranges there between. In certain aspects, the peptide fibril formed by the self-assembling peptides may have a length of about, at least about, or at most about 0.01, 0.1, 1.0, 10, 20, 50, 100, 500, or 1000 μm or nm (or any range or value derivable therein).

In certain aspects, the peptide fibril has a molecular weight of at least 1,000, 5,000, 10,000, 100,000 da to 1×10⁶, 1×10⁷, 7×10⁸ da, including all values and ranges there between. In other aspects the self-assembling peptides form a beta-sheet rich fibril.

In further aspects, the self-assembling peptide comprises an amino acid sequence (from amino to carboxy terminus) having the amino acid sequence of FKFEFKFE (SEQ ID NO:1); QQKFQFQFEQQ (SEQ ID NO:2); KFQFQFE (SEQ ID NO:3); QQRFQFQFEQQ (SEQ ID NO:4); QQRFQWQFEQQ (SEQ ID NO:5); FEFEFKFKFEFEFKFK (SEQ ID NO:6); QQRFEWEFEQQ (SEQ ID NO:7); QQXFXWXFQQQ (SEQ ID NO:8) (Where X denotes ornithine); FKFEFKFEFKFE (SEQ ID NO:9); FKFQFKFQFKFQ (SEQ ID NO:10); AEAKAEAKAEAKAEAK (SEQ ID NO:11); AEAEAKAKAEAEAKAK (SEQ ID NO:12); AEAEAEAEAKAKAKAK (SEQ ID NO:13); RADARADARADARADA (SEQ ID NO:14); RARADADARARADADA (SEQ ID NO:15); SGRGYBLGGQGAGAAAAAGGAGQGGYGGLGSQG (SEQ ID NO:16); EWEXEXEXEX (SEQ ID NO:17) (Where X=V, A, S, or P); WKXKXKXKXK (SEQ ID NO:18) (Where X=V, A, S, or P); KWKVKVKVKVKVKVK (SEQ ID NO:19); LLLLKKKKKKKKLLLL (SEQ ID NO:20); VKVKVKVKVDPPTKVKVKVKV (SEQ ID NO:21); VKVKVKVKVDPPTKVKTKVKV (SEQ ID NO:22); KVKVKVKVKDPPSVKVKVKVK (SEQ ID NO:23); VKVKVKVKVDPPSKVKVKVKV (SEQ ID NO:24); or VKVKVKTKVDPPTKVKTKVKV (SEQ ID NO:25). In certain aspects the cocaine hapten is coupled to the self-assembling peptide by a linker. In a further aspect the linker is a peptide linker, which includes a poly-glycine linker, e.g., a GGG linker. In certain aspects the linker is coupled to the carboxy or amino terminus of the self-assembling peptide. In certain embodiments the self-assembling peptide with poly-glycine linker has an amino acid sequence of GGGFKFEFKFE (SEQ ID NO:28). In a further aspect the carboxy and/or amino terminus can be chemically modified.

In certain aspects, more than one self-assembling peptide is present in a peptide fibril. In certain aspects the cocaine hapten is covalently coupled to the self-assembling peptide or peptide fibril. In a further aspect the cocaine antigen is coupled to the peptide fibril via a linker. In certain aspects, the polypeptides are covalently coupled to the peptide fibril via a SGSG (SEQ ID NO:26) linker or a GGAAY (SEQ ID NO:27) linker. In further embodiments, the linker may be at least, at most or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids (or any range or value derivable therein).

In further aspects, peptides of the invention can be comprised of L-amino acids, D-amino acids, or a combination thereof.

In a further aspect, the cocaine hapten is covalently coupled to the self-assembling peptide. In a further aspect, the antigen is covalently coupled to the N and/or C terminus of the self-assembling peptide. In still further aspects, the antigen is covalently coupled to the carboxy terminus of the self-assembling peptide. In certain aspects the ratio of antigen to self-assembling peptide is 1:1000, 1:100:1:10, or 1:1, including all values and ranges there between.

Certain embodiments are directed to methods of inducing an immune response comprising administering an antigenic fibril, comprising a self-assembling peptide coupled to a cocaine hapten, to a subject in an amount sufficient to induce an immune response.

Further embodiments are directed to methods of treating a subject having or at risk of developing a cocaine use disorder that can be treated by inducing an immune response comprising administering to the subject an effective amount of a composition described herein. In other aspects, an antibody that specifically binds the cocaine hapten can be administered to a subject.

Methods may involve administering to the patient or subject at least or at most 1, 2, 3, 4, 5, 6, 7, 8, 9, 10 or more doses of a pharmaceutical composition or a composition described herein. A dose may be a composition comprising about, at least about, or at most about 0.01, 0.1, 1.0, 10, 50, 100, 500, 1000, 5000, to 10,000 milligrams (mg) or micrograms (mcg) or μg/ml or micrograms/ml or mM or μM (or any value or range there between) of an immunogenic fibril as described herein.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of ‘one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

“Patient,” “subject,” or “individual” refers to a mammal (e.g., human, primate, dog, cat, bovine, ovine, porcine, equine, mouse, rat, hamster, rabbit, or guinea pig). In particular aspects, the patient, subject, or individual is a human.

“Inhibiting” or “reducing” or any variation of these terms includes any measurable decrease or complete inhibition to achieve a desired result. The terms “promote” or “increase” or any variation of these terms includes any measurable increase or production of a protein or molecule to achieve a desired result.

“Effective” or any variation of this term means adequate to accomplish a desired, expected, or intended result. The result may include, but is not limited to any measurable change in an activity, production, a disease, a condition, or a symptom.

“Treating” or any variation of this term includes any measurable improvement in a disease, condition, or symptom that is being treated or is associated with the disease, condition, or symptom being treated.

“Preventing” or any variation of this term means to slow, stop, or reverse progression toward a result. The prevention may be any slowing of the progression toward the result.

“Analogue” and “analog,” when referring to a compound, refers to a modified compound wherein one or more atoms have been substituted by other atoms, or wherein one or more atoms have been deleted from the compound, or wherein one or more atoms have been added to the compound, or any combination of such modifications. Such addition, deletion, or substitution of atoms can take place at any point, or multiple points, along the primary structure comprising the compound.

“Derivative,” in relation to a parent compound, refers to a chemically modified parent compound or an analog thereof, wherein at least one substituent is not present in the parent compound or an analog thereof. One such non-limiting example is a parent compound which has been covalently modified. Typical modifications are amides, carbohydrates, alkyl groups, acyl groups, esters, pegylations and the like.

A “therapeutically equivalent” compound is one that has essentially the same effect in the treatment of a disease or condition as one or more other compounds. A compound that is therapeutically equivalent may or may not be chemically equivalent, bioequivalent, or generically equivalent.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1 Structures of cocaine and representative reported haptens.

FIG. 2 Chemical structures of representative cocaine haptens with linkers at P3 position.

FIG. 3 Chemical structures of representative nanofiber-based cocaine vaccines.

FIG. 4 General chemical structures of cocaine haptens.

FIG. 5 General chemical structures of nanofiber-based cocaine vaccines.

FIG. 6 Non-limiting cocaine haptens with linkers at P3 position and synthetic methods.

FIG. 7 Schematic of synthesis for cocaine-KFE8 conjugates. R1 and R2 indicate orthogonal chemical groups and n indicates linker length. Non-limiting example chemistries are shown in the table.

FIG. 8 Non-limiting examples of haptens with linkers at P3 position and the synthetic methods.

FIG. 9 Non-limiting examples of haptens with linkers at P5 position and the synthetic methods.

FIG. 10 Synthesis of novel cocaine haptens modified at the P3 (cocaine N-site) site and conjugation to the self-assembling peptide domain KFE8. Reagents and conditions: (a) Saturated NaHCO₃ aqueous solution, CH₂Cl₂, rt, 1 h, 96%; (b) CH₃CH(Cl)OCOCl, 1,2-dichloroethane, reflux, 7 h, 91%; (c) MeOH, reflux, 8 h, 90%; (d) BnOCOCH₂CH₂CH₂CH₂Br, K₂CO₃, KI, acetone, 24 h, 73%; (e) H₂, Pd-C, MeOH, rt, 12 h, 75%; (f) 20% piperidine in DMF, rt; (g) 4, HBTU, DIEA, HOBt, DIC, DMF, rt, 2.5 h; (h) Trifluoroacetic acid/H₂O/Triisopropylsilane (95%/2.5%/2.5%), rt, 1.5 h.

FIG. 11 Self-assembly and secondary structure analysis of CocKFE8 conjugates. (A) Nanofibers of KFE8 and (B) CocKFE8 as observed by transmission electron microscopy. Scale bar=50 nm. (C) CD spectra of CocKFE8 conjugate showing β-sheet rich secondary structure. Peptide concentration was 0.5 mM in water.

FIG. 12 Immunization regime, antibody responses, and suppression of cocaine-evoked hyperactivity in mice. (A) CocKFE8 nanofibers induced adjuvant-free antibody responses in mice as measured by ELISA and mice with absorbance values higher than the control (PBS-treated) group mean plus three times the standard deviation (i.e. >0.05 absorbance, represented by the dashed line) were defined as CocKFE8 responders (*p<0.05 vs. controls, ̂p<0.05 vs. CocKFE8 non-responders). (B) Vaccination with CocKFE8 did not alter spontaneous locomotor activity (p>0.05). (D) Mice responding to CocKFE8 nanofiber vaccines show blunted cocaine-evoked locomotor activity (*p<0.05 vs. control). The time course of peripheral activity is divided into 15 min time bins across the 60 min session for both spontaneous and cocaine-evoked locomotor activity. The mean total spontaneous and cocaine-evoked peripheral activity (counts/60 min) (±SEM) is represented in the inset for FIG. 12C and FIG. 12E, respectively.

FIG. 13 Significant negative correlation between antibody titers and cocaine-evoked hyperactivity measures for individual mice (r=−0.395; p<0.05).

FIG. 14 Conceptual overview of use of self-assembling peptide-cocaine conjugates to treat and prevent cocaine use disorder and manage acute cocaine overdose.

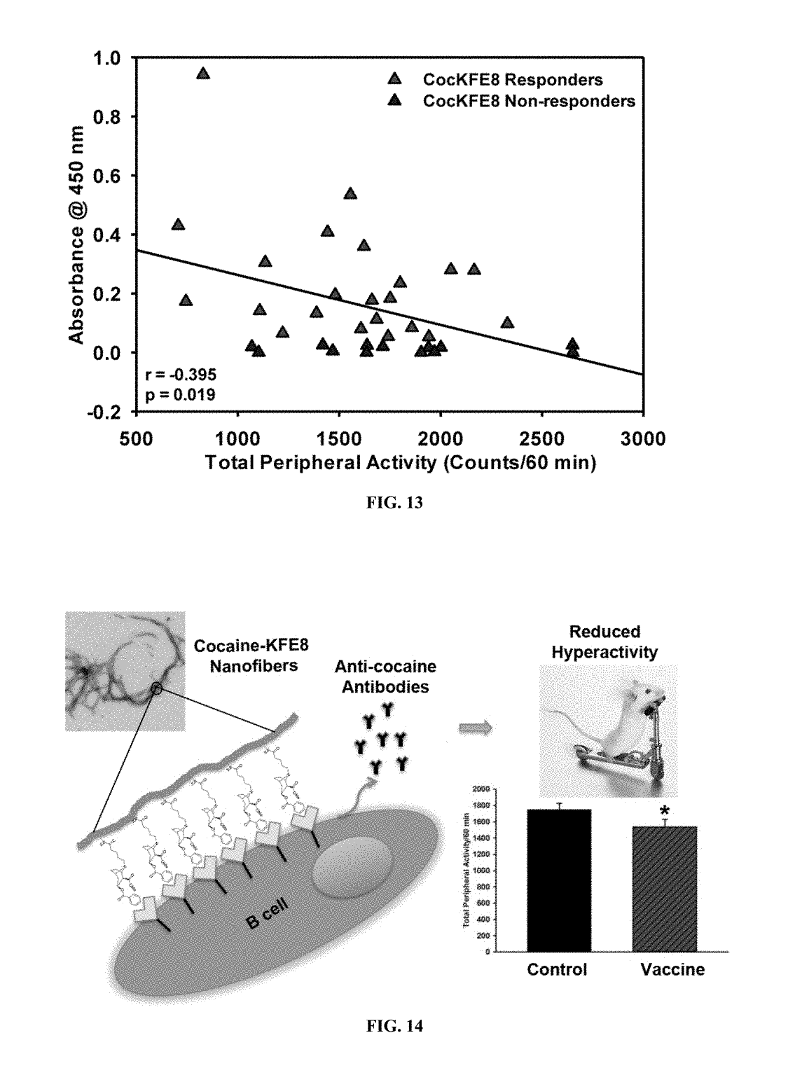

### DESCRIPTION

A rapidly growing area in vaccine development and immunology is the design of nano-scaffolds with high surface-area-to-volume ratio for multivalent antigen presentation (Smith et al., 2013). Multivalent interactions between the antigen and immune cell receptors provide a significant enhancement in binding affinity and specificity leading to strong adaptive immune responses (Englund et al., 2012). Compared to other macromolecular architectures such as branched polymers, dendrimers, virus-like particles, and DNA, peptide-based scaffolds offer the advantage of ease of synthesis, characterization, chemical diversity, biocompatibility, and control over the primary sequence to obtain morphologies such as nanotubes, fibrils, nanoparticles, and vesicles (Petkau-Milroy et al., 2013; Kushner et al., 2011). Furthermore, relatively long peptides can be realized using totally synthetic strategies whose purity and identity can be confirmed using mass-spectrometry, a key advantage for regulatory approval compared to heterogeneous mixtures.

Self-assembling peptides that assemble into β-sheet rich nanofibers in physiological buffers have been investigated recently as immune adjuvants for vaccine development (Rudra et al., 2010; Wen et al., 2015; Chen et al., 2013). The ability of the nanofibers to self-assemble is maintained when other bioactive molecules are covalently attached at either terminus and presented on the surface of the nanofibers in high-density multivalent arrays (Wen et al., 2015). When peptide or protein antigens are linked to self-assembling peptides, the resulting nanofibers are highly immunogenic and elicit strong antibody and cellular responses in mice without the need for any exogenous adjuvants (Rudra et al., 2010). The adjuvanting activity of the nanofibers is not restricted to a particular primary sequence, route of immunization, or mouse strain (Rudra et al., Acs Nano, 2012; Huang et al., 2012). Vaccination with peptide nanofibers bearing disease specific epitopes has been shown to be protective in mouse models of malaria (Rudra et al., Biomaterials, 2012), cancer (Huang et al., 2012), and influenza (Chesson et al., 2014).

However, questions regarding the mechanism of action of nanofiber vaccines, the in vivo fate and toxicity of the vaccine, and the efficacy and applicability of nanofiber vaccines to illicit a response to cocaine have not been answered. Thus, there remains a need for further development of therapeutics for cocaine use disorder and for the management of acute overdose.

Herein, certain embodiments are directed to chemically defined self-adjuvanting cocaine vaccines composed of novel cocaine haptens and self-assembling peptide domains. Immunological evaluation of cocaine nanofiber vaccines disclosed herein show that anti-cocaine antibodies were elicited and counteracted the behavioral effects of cocaine in vivo. These totally synthetic and multivalent nanofibers with well-defined chemical composition represent the first generation of adjuvant-free cocaine vaccines.

### I. Synthesis of Cocaine Haptens

Cocaine haptens were produced for use as immune adjuvants for antibody production against small drug molecules. Several novel cocaine analogs with various chemical linker moieties and linker lengths were synthesized.

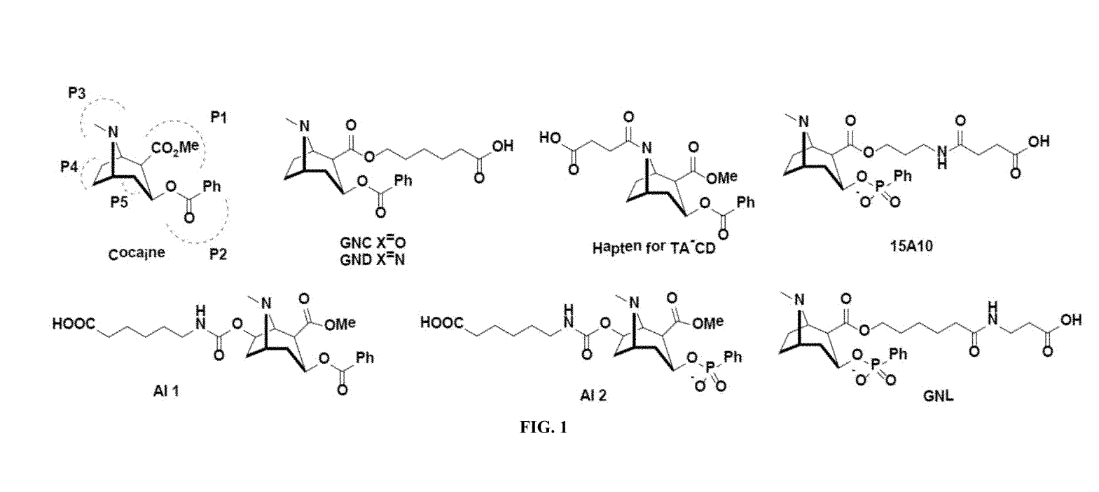

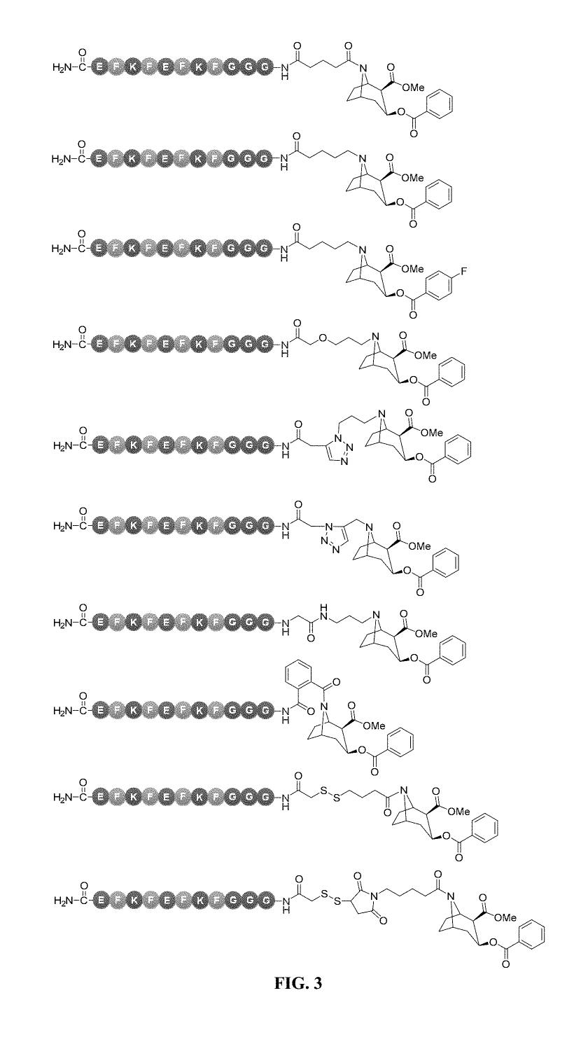

To the best of our knowledge, previously reported haptens have been designed with a linker moiety positioned at the methyl ester (P1), benzoate (P2), N-methyl amino (P3) and two-carbon bridge (P4) moieties of cocaine. See FIG. 1. However, placing the linker at these positions may also decrease efficient antibody binding due to the burying of the key epitopes and the important binding region. As a matter of fact, most of these haptens reported in did not reach clinical trials except TA-CD with a linker at the P3 position. However, optimization of linkers at the P3 position has not been extensively reported. To test for an improved immune response by positioning the linker at positions such as P3 and P5 distal from the key epitopes and binding region present in cocaine, several cocaine haptens were produced. Cocaine haptens disclosed therein include, but are not limited to, those of Formula I (FIG. 4):

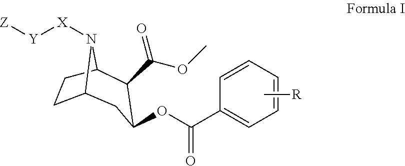

wherein
X is —CH₂—, —CO—, or —CS—;
Y is substituted alkane, alkene, alkyne, or aromatic rings;
Z is —CO₂H—, —OH, N₃, —NH₂, —CHO, —SH, —CH═CH₂, —C≡CH, or

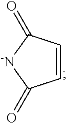

and
and R is hydrogen, hydroxyl, nitro, mercapto, cyano, azido, alkyl, heteroalkyl, alkoxy, halogen, amino, oxo, or alkylsulfonyl. In certain aspects R is in particular —F, —Cl, or —Br. In certain aspects R is at the 2, 3, 4, 5, or 6 position.

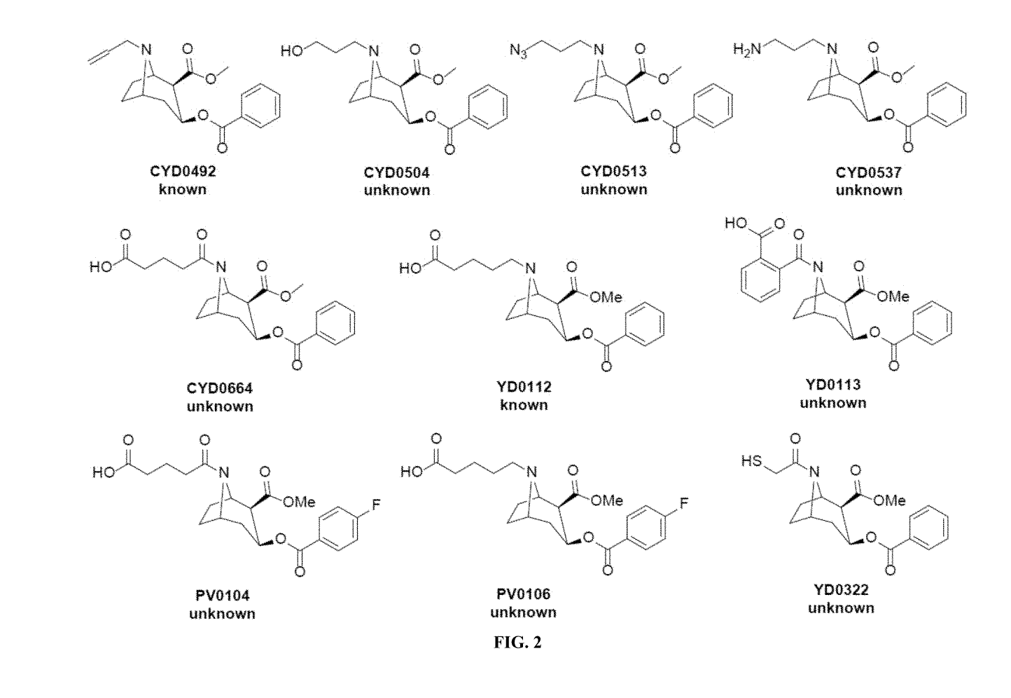

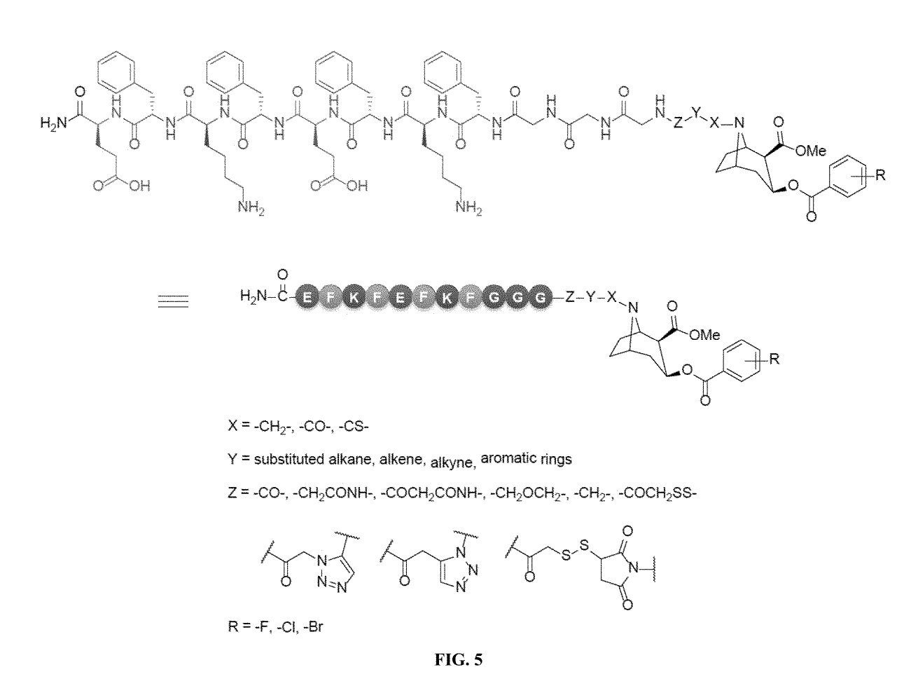

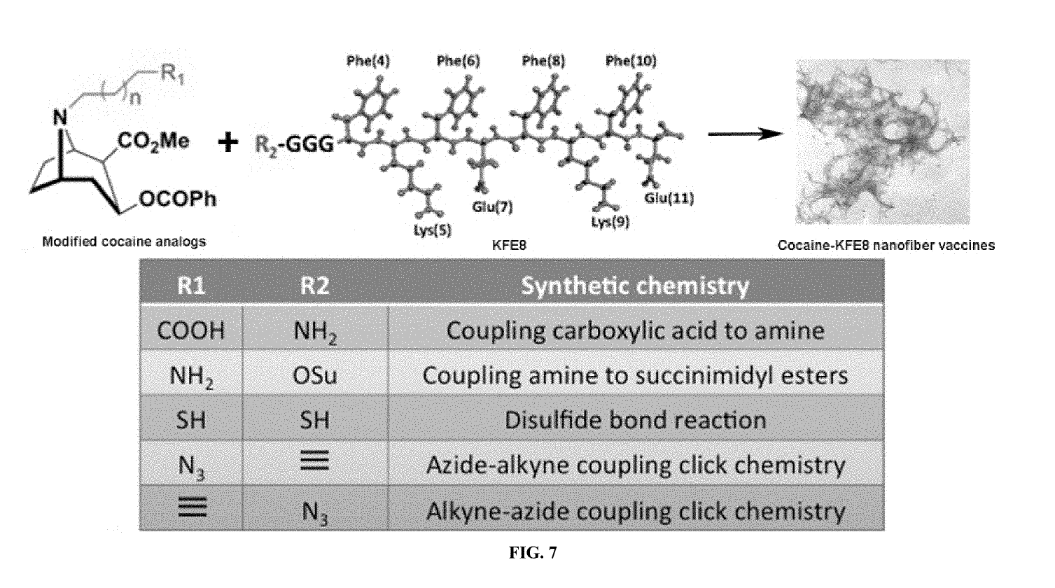

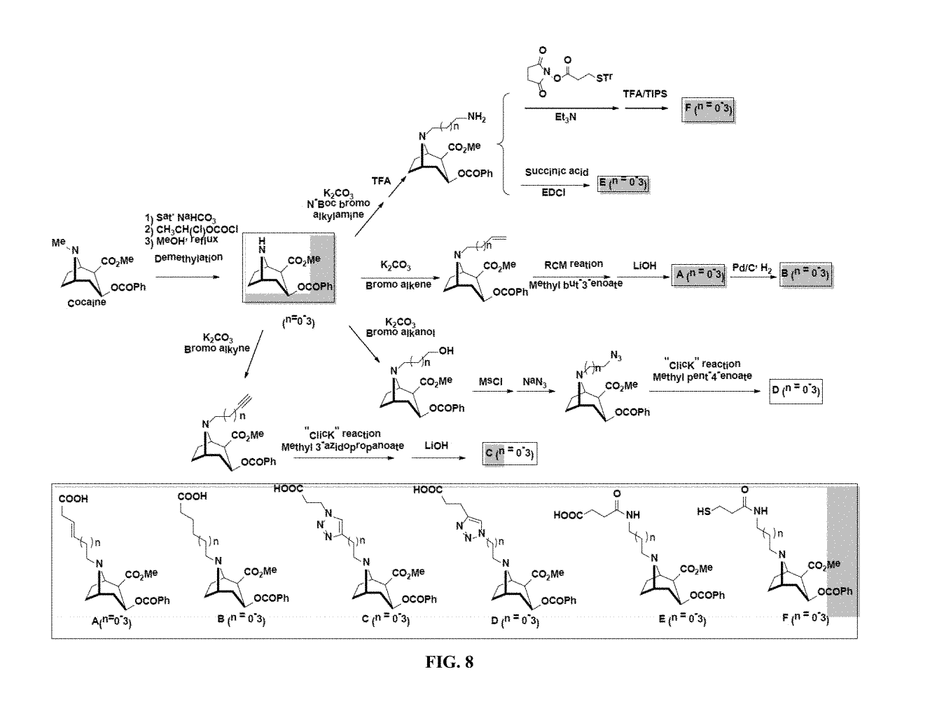

In some embodiments, the cocaine haptens include a cocaine hapten as described in FIG. 2. In some embodiments, the cocaine haptens are included in several types of new haptens with different linkers at the P3 position described in series A-F (n=1-3) of FIG. 6. Various alkyl chains as linkers may be introduced at the tropane nitrogen. In certain aspects the alkyl chain is attached to the tropane nitrogen via firm C—N bond, which is more stable in vivo. In a further aspect the alkyl chain can mimic the N-methyl functionality of cocaine, and the native characteristic of N-methyl can be retained as much as possible. Synthetic approaches to these haptens are described below and are depicted in FIGS. 6, 8, and 9. Compounds series A, B, C, D, E and F as shown in FIG. 6, were synthesized using the key intermediate norcocaine prepared from cocaine by N-demethylation. Functionalities may be added such as, but not limited to, acid, amino, azide, alkyne, or mercapto groups. Synthesis of selected cocaine haptens are described in detail below.

Various chemical definitions related to such compounds are provided as follows.

As used herein, the term “nitro” means —NO₂; the term “halo” designates —F, —Cl, —Br or —I; the term “mercapto” means —SH; the term “cyano” means —CN; the term “azido” means —N₃; the term “silyl” means —SiH₃, and the term “hydroxyl” means —OH.

The term “alkyl,” by itself or as part of another substituent, means, unless otherwise stated, a linear (i.e. unbranched) or branched carbon chain, which may be fully saturated, mono- or polyunsaturated. An unsaturated alkyl group is one having one or more double bonds or triple bonds. Saturated alkyl groups include those having one or more carbon-carbon double bonds (alkenyl) and those having one or more carbon-carbon triple bonds (alkynyl). The groups, —CH₃ (Me), —CH₂CH₃ (Et), —CH₂CH₂CH₃ (n-Pr), —CH(CH₃)₂ (iso-Pr), —CH₂CH₂CH₂CH₃ (n-Bu), —CH(CH₃)CH₂CH₃ (sec-butyl), —CH₂CH(CH₃)₂ (iso-butyl), —C(CH₃)₃ (tert-butyl), —CH₂C(CH₃)₃ (neo-pentyl), are all non-limiting examples of alkyl groups.

The term “heteroalkyl,” by itself or in combination with another term, means, unless otherwise stated, a linear or branched chain having at least one carbon atom and at least one heteroatom selected from the group consisting of O, N, S, P, and Si. In certain embodiments, the heteroatoms are selected from the group consisting of 0 and N. The heteroatom(s) may be placed at any interior position of the heteroalkyl group or at the position at which the alkyl group is attached to the remainder of the molecule. Up to two heteroatoms may be consecutive. The following groups are all non-limiting examples of heteroalkyl groups: trifluoromethyl, —CH₂ F, —CH₂ Cl, —CH₂ Br, —CH₂ OH, —CH₂ OCH₃, —CH₂ OCH₂ CF₃, —CH₂OC(O)CH₃, —CH₂ NH₂, —CH₂ NHCH₃, —CH₂ N(CH₃)₂, —CH₂CH₂Cl, —CH₂CH₂OH, CH₂CH₂OC(O)CH₃, —CH₂CH₂ NHCO₂C(CH₃)₃, and —CH₂ Si(CH₃)₃.

The term “alkoxy” means a group having the structure —OR′, where R′ is an optionally substituted alkyl or cycloalkyl group. The term “heteroalkoxy” similarly means a group having the structure —OR, where R is a heteroalkyl or heterocyclyl.

The term “amino” means a group having the structure —NR′R″, where R′ and R″ are independently hydrogen or an optionally substituted alkyl, heteroalkyl, cycloalkyl, or heterocyclyl group. The term “amino” includes primary, secondary, and tertiary amines.

The term “oxo” as used herein means an oxygen that is double bonded to a carbon atom.

The term “alkylsulfonyl” as used herein means a moiety having the formula —S(O₂)—R′, where R′ is an alkyl group. R′ may have a specified number of carbons (e.g. “C₁₋₄ alkylsulfonyl”)

The term “pharmaceutically acceptable salts,” as used herein, refers to salts of compounds of this invention that are substantially non-toxic to living organisms. Typical pharmaceutically acceptable salts include those salts prepared by reaction of a compound of this invention with an inorganic or organic acid, or an organic base, depending on the substituents present on the compounds of the invention.

Non-limiting examples of inorganic acids which may be used to prepare pharmaceutically acceptable salts include: hydrochloric acid, phosphoric acid, sulfuric acid, hydrobromic acid, hydroiodic acid, phosphorous acid and the like. Examples of organic acids which may be used to prepare pharmaceutically acceptable salts include: aliphatic mono- and dicarboxylic acids, such as oxalic acid, carbonic acid, citric acid, succinic acid, phenyl-heteroatom-substituted alkanoic acids, aliphatic and aromatic sulfuric acids and the like. Pharmaceutically acceptable salts prepared from inorganic or organic acids thus include hydrochloride, hydrobromide, nitrate, sulfate, pyrosulfate, bisulfate, sulfite, bisulfate, phosphate, monohydrogenphosphate, dihydrogenphosphate, metaphosphate, pyrophosphate, hydroiodide, hydro fluoride, acetate, propionate, formate, oxalate, citrate, lactate, p-toluenesulfonate, methanesulfonate, maleate, and the like.

Suitable pharmaceutically acceptable salts may also be formed by reacting the agents of the invention with an organic base such as methylamine, ethylamine, ethanolamine, lysine, ornithine and the like. Pharmaceutically acceptable salts include the salts formed between carboxylate or sulfonate groups found on some of the compounds of this invention and inorganic cations, such as sodium, potassium, ammonium, or calcium, or such organic cations as isopropylammonium, trimethylammonium, tetramethylammonium, and imidazolium.

Additional examples of pharmaceutically acceptable salts and their methods of preparation and use are presented in Handbook of Pharmaceutical Salts: Properties, Selection and Use (2002), which is incorporated herein by reference.

Generally, all commercially available starting materials and solvents were reagent grade and used without further purification. The key intermediate (CYD-4-90) and benzyl 5-bromopentanoate were synthesized following the reported procedures (Center for Behavioral Health Statistics and Quality, 2015; Shen et al., 2012, respectively). Reactions were performed under a nitrogen atmosphere in dry glassware with magnetic stirring. Preparative column chromatography was performed using silica gel 60, particle size 0.063-0.200 mm (70-230 mesh, flash). Analytical TLC was carried out employing silica gel 60 F254 plates (Merck, Darmstadt). The developed chromatogram was visualized by UV detection (254 nm). NMR spectra were recorded on a Bruker-300 (¹H, 300 MHz; ¹³C, 75 MHz) spectrometer. ¹H and ¹³C NMR spectra were recorded with TMS as an internal reference. Chemical shifts downfield from TMS were expressed in ppm, and J values were given in Hz. High-resolution mass spectra (HRMS) were obtained from Thermo Fisher LTQ Orbitrap Elite mass spectrometer. Parameters include the following: nano ESI spray voltage was 1.8 kV, capillary temperature was 275° C., and the resolution was 60000; ionization was achieved by positive mode. Purity of final compounds was determined by analytical HPLC, which was carried out on a Shimadzu HPLC system (model: CBM-20A LC-20AD SPD-20A UV/vis). HPLC analysis conditions: Waters pondapak C18 (300 mm×3.9 mm), flow rate 0.5 mL/min, UV detection at 270 and 254 nm, linear gradient from 10% acetonitrile in water to 100% acetonitrile in 20 min, followed by 30 min of the last-named solvent.

Scheme I illustrating synthesis of CYD-5-4.

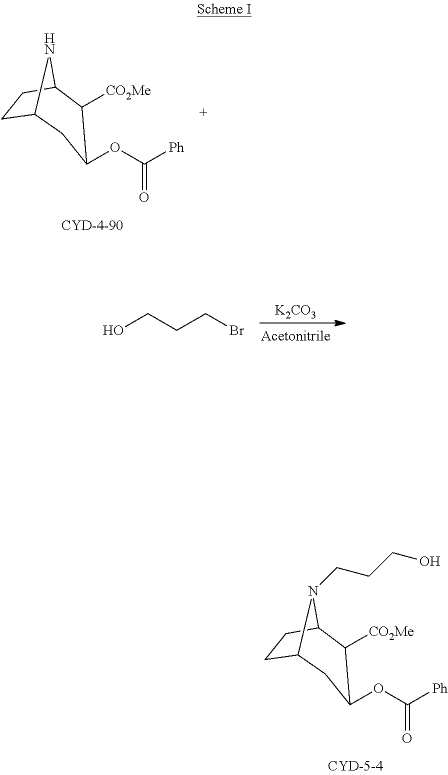

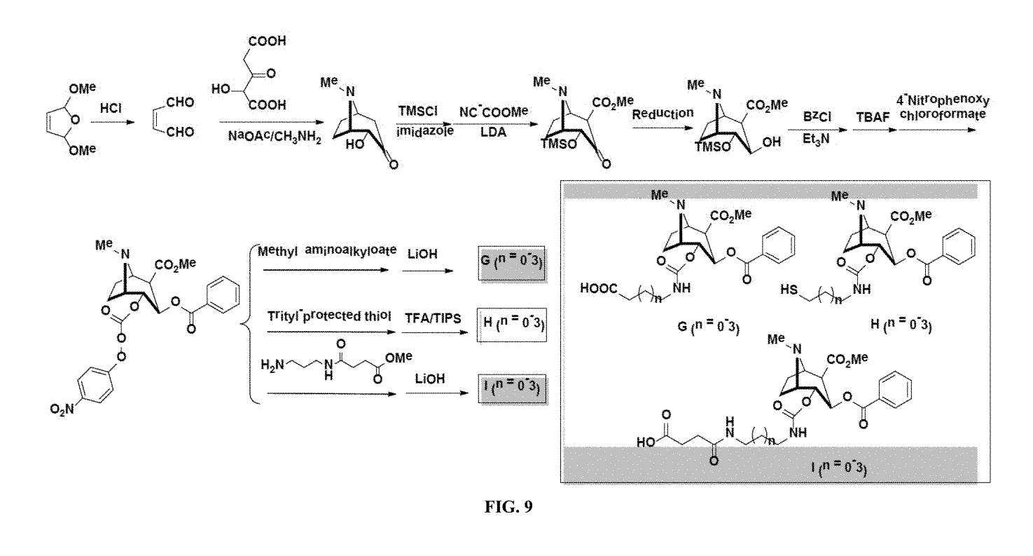

CYD-4-90 (norcocaine) was synthesized by N-demethylation of cocaine using alpha-choroethyl chloroformate (Zhou et al., 2003) in a total yield of 79% (three steps) (See steps a-c in FIG. 10).

A mixture of CYD-4-90 (1000 mg, 3.46 mmol), 3-bromo-propan-1-ol (0.45 mL, 5.19 mmol), and K₂CO₃ (716 mg, 5.19 mmol) was refluxed in acetone (40 mL) for 16 h. The solvent was removed, and the residue was dissolved in ice-cold ammonia solution (2% v/v). The aqueous solution was extracted with dichloromethane and dried over anhydrous Na₂SO₄, filtered, and evaporated to give an oily residue. The residue was purified using silica gel column; elution with 5% Et₃N in ether acetate afforded the desired product CYD-5-4 as a colorless amorphous gel (600 mg, 67%). ¹H NMR (600 MHz, CDCl₃): δ 8.00 (m, 2H), 7.52 (m, 1H), 7.40 (t, 2H, J=7.8 Hz), 5.26 (m, 1H), 4.68 (br s, 1H), 3.77 (m, 2H), 3.72 (s, 3H), 3.64 (d, 1H, J=4.2 Hz), 3.49 (d, 1H, J=3.0 Hz), 3.08 (dd, 1H, J=3.0 Hz, 6.0 Hz), 2.53 (m, 1H), 2.40 (m, 2H), 2.05 (m, 2H), 1.92 (m, 1H), 1.71 (m, 3H), 1.55 (m, 1H). ¹³C NMR (150 MHz, CDCl₃) δ 167.1, 162.2, 129.3, 126.4, 125.9 (2C), 124.6 (2C), 63.2, 60.1, 59.9, 55.3, 49.3, 47.9, 46.6, 31.6, 26.0, 22.1, 21.4.

Scheme II illustrating synthesis of CYD-5-13.

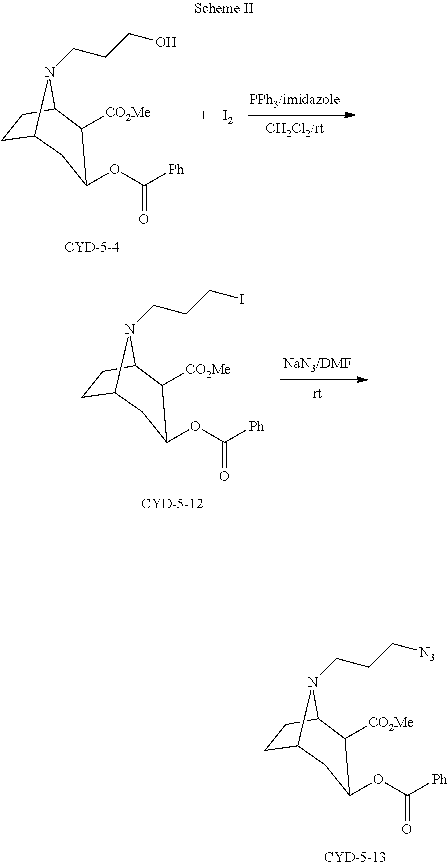 [SMILES: COC(=O)C1C(OC(=O)c2ccccc2)CC2CCC1N2CCCO ; "I2" ; COC(=O)C1C(OC(=O)c2ccccc2)CC2CCC1N2CCCI ; COC(=O)C1C(OC(=O)c2ccccc2)CC2CCC1N2CCCN=[N+]=[N-]]

To a solution of CYD-5-4 (200 mg, 0.57 mmol) in dichloromethane (30 mL) was added I₂ (321 mg, 1.26 mmol), PPh₃ (332 mg, 1.26 mmol), and imidazole (98 mg, 1.43 mmol) at room temperature (rt), and the resulting mixture was refluxed for 5 h. The reaction solution was washed with sat. Na₂S₂O₃ (aq) and dried over anhydrous Na₂SO₄, filtered, and evaporated to give an oily residue. The residue was purified using silica gel column; elution with 20% EtOAc in hexane afforded the desired product CYD-5-12 as a colorless amorphous gel (110 mg, 42%).

A mixture of CYD-5-12 (100 mg, 0.22 mmol) and NaN₃ (56 mg, 0.87 mmol) was stirred in DMF (3 mL) at rt for 18 h at the dark. The reaction mixture was then diluted with water and extracted with dichloromethane. The extract was washed with brine and dried over anhydrous Na₂SO₄, filtered, and evaporated to give an oily residue. The residue was purified using silica gel column; elution with 16% EtOAc in hexane afforded the desired product CYD-5-13 as a colorless amorphous gel (65 mg, 80%). ¹H NMR (600 MHz, CDCl₃): δ 8.04 (m, 2H), 7.55 (m, 1H), 7.44 (m, 2H), 5.26 (m, 1H), 3.72 (s, 3H), 3.67 (m, 1H), 3.41 (t, 2H, J=5.4 Hz), 3.34 (m, 1H), 3.34 (m, 1H), 2.44 (dt, 1H, J=2.4 Hz, 9.6 Hz), 2.35 (m, 2H), 2.08 (m, 1H), 2.01 (m, 1H), 1.88 (m, 1H), 1.75 (m, 2H), 1.66 (m, 2H). ¹³C NMR (150 MHz, CDCl₃) δ 170.6, 166.1, 132.9, 130.3, 129.7 (2C), 128.3 (2C), 67.2, 62.7, 60.6, 51.3, 50.3, 49.5, 49.0, 35.7, 28.4, 26.1, 25.5.

Scheme III illustrating synthesis of CYD-5-37.

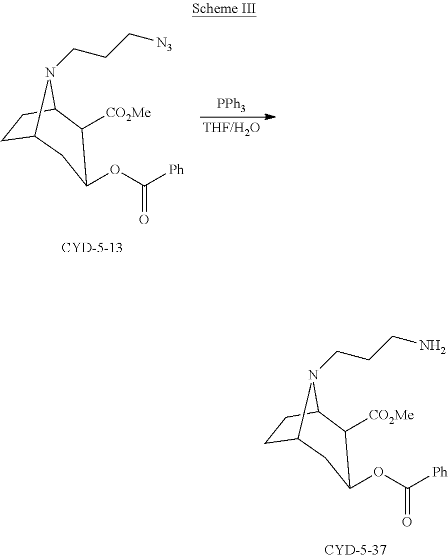 [SMILES: COC(=O)C1C(OC(=O)c2ccccc2)CC2CCC1N2CCCN=[N+]=[N-] ; COC(=O)C1C(OC(=O)c2ccccc2)CC2CCC1N2CCCN ; "PPh3"]

A mixture of CYD-5-13 (60 mg, 0.16 mmol) and PPh₃ (50 mg, 0.19 mmol) was stirred in a mixture solution of THF (5 mL) and H₂O (0.75 mL) at rt for 24 h. The reaction mixture was then diluted with water and extracted with dichloromethane. The extract was washed with brine and dried over anhydrous Na₂SO₄, filtered, and evaporated to give an oily residue. The residue was purified using silica gel column; elution with 5% MeOH in CH₂Cl₂ afforded the desired product CYD-5-37 as a colorless amorphous gel (65 mg, 80%). ¹H NMR (600 MHz, CDCl₃): δ 8.02 (m, 2H), 7.53 (m, 1H), 7.41 (m, 2H), 5.25 (m, 1H), 3.70 (s, 3H), 3.68 (m, 1H), 3.38 (d, 1H, J=2.4 Hz), 3.04 (dd, 1H, J=3.6 Hz, 5.4 Hz), 2.79 (br s, 2H), 2.39 (m, 2H), 2.30 (m, 1H), 2.04 (m, 2H), 1.88 (m, 1H), 1.74 (m, 4H), 1.55 (m, 2H). ¹³C NMR (150 MHz, CDCl₃) δ 170.8, 166.1, 132.9, 130.3, 129.7 (2C), 128.3 (2C), 67.3, 63.0, 59.9, 51.3, 50.8, 50.3, 40.4, 35.6, 32.1, 25.8, 25.6.

Scheme IV illustrating synthesis of CYD-6-64.

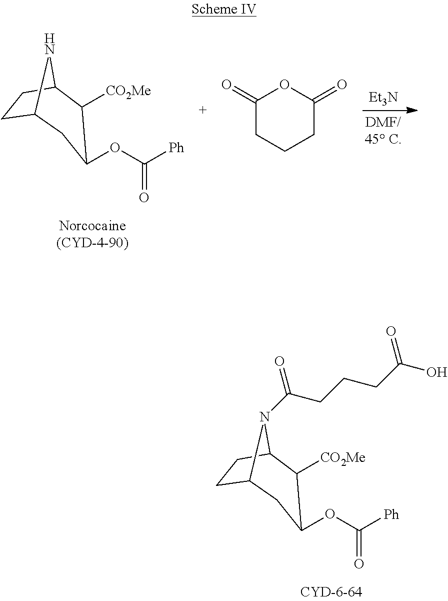 [SMILES: "Et3N" ; COC(=O)C1C2CCC(CC1OC(=O)c1ccccc1)N2 ; O=C1CCCC(=O)O1 ; COC(=O)C1C(OC(=O)c2ccccc2)CC2CCC1N2C(=O)CCCC(=O)O]

To a solution of norcocaine (500 mg, 1.73 mmol) and glutaric anhydrous (394 mg, 3.46 mmol) in DMF (5 mL) was added Et₃N (502 μL, 3.46 mmol) at rt, and the resulting mixture was stirred at 45° C. for 24 h. The reaction mixture was then diluted with water and extracted with dichloromethane. The extract was washed with brine and dried over anhydrous Na₂SO₄, filtered, and evaporated to give an oily residue. The residue was purified using silica gel column; elution with 10% MeOH in CH₂Cl₂ afforded the desired product CYD-6-64 as a colorless amorphous gel (650 mg, 93%). ¹H NMR (600 MHz, CDCl₃): δ 10.0 (br s, 1H), 7.95 (m, 2H), 7.55 (m, 1H), 7.42 (m, 2H), 5.51 (m, 1H), 5.08 and 4.95 (m, 1H), 4.54 and 4.41 (m, 1H), 3.66 and 3.65 (d, 3H), 3.22 and 3.14 (m, 1H), 3.44 (m, 4H), 2.34 and 2.24 (m, 1H), 2.07 (m, 2H), 1.97 (m, 5H). ¹³C NMR (150 MHz, CDCl₃) δ 177.4, 177.2, 173.5, 170.1, 169.1, 165.7 (2C), 163.3, 133.3, 129.6 (3C), 128.4 (2C), 66.4, 66.3, 55.4, 53.6, 52.9, 52.0, 51.8, 51.6, 50.4, 49.1, 48.6, 36.8, 34.3, 33.2, 32.9, 32.4, 32.0, 31.7, 30.9, 29.6, 29.0, 28.4, 26.8, 26.4, 20.2 (2C), 19.9.

Scheme illustrating synthesis of CYD-4-92.

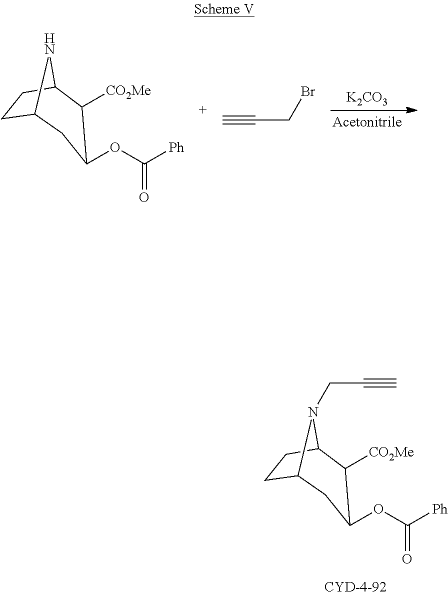

A mixture of norcocaine (700 mg, 2.42 mmol), propargyl bromide (0.26 mL, 2.90 mmol), and K₂CO₃ (401 mg, 2.90 mmol) was refluxed in acetonitrile (40 mL) for 24 h. The solvent was removed, and the residue was dissolved in ice-cold ammonia solution (2% v/v). The aqueous solution was extracted with ether and dried over anhydrous Na₂SO₄, filtered, and evaporated to give an oily residue. The residue was purified using silica gel column; elution with 20% EtOAc in hexanes afforded the desired product CYD-4-92 as a colorless amorphous gel (600 mg, 76%). ¹H NMR (300 MHz, CDCl₃): δ 8.03 (d, 2H, J=7.8 Hz), 7.53 (t, 1H, J=6.9 Hz), 7.41 (m, 2H), 5.76 (m, 1H), 5.26 (m, 1H), 5.10 (m, 2H), 3.70 (s, 3H), 3.67 (m, 1H), 3.36 (m, 1H), 3.04 (m, 1H), 2.45 (m, 1H), 2.06 (m, 2H), 1.87 (m, 1H), 1.76 (m, 2H).

Scheme VI illustrating synthesis of 5-((1R,2R,3S,5S)-3-(benzoyloxy)-2-(methoxycarbonyl)-8-azabicyclo[3.2.1]octan-8-yl)pentanoic acid (YD0112).

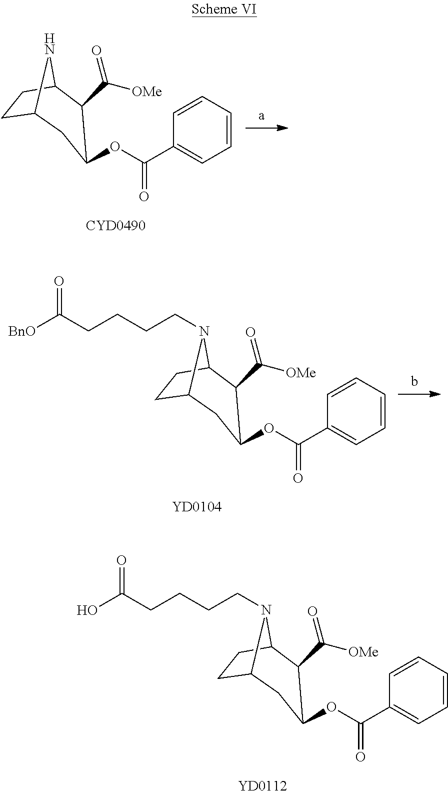 [SMILES: COC(=O)[C@@H]1C2CCC(C[C@@H]1OC(=O)c1ccccc1)N2 ; "CYD0490" ; COC(=O)[C@@H]1C2CCC(C[C@@H]1OC(=O)c1ccccc1)N2CCCCC(=O)OCc1ccccc1 ; "YD0104" ; COC(=O)[C@@H]1C2CCC(C[C@@H]1OC(=O)c1ccccc1)N2CCCCC(=O)O ; "YD0112"]

Reagents and conditions: (a) BnOCOCH₂CH₂CH₂CH₂Br, K₂CO₃, KI, acetone, 12 h; and (b) H₂, Pd-C, MeOH, rt, overnight. To a stirring solution of CYD0490 (279 mg, 0.97 mmol) and K₂CO₃ (268 mg, 1.94 mmol) in acetone (10 mL) was added benzyl 5-bromopentanoate (314 mg, 1.16 mmol) and KI (161 mg, 0.97 mmol) at room temperature. The resulting mixture was refluxed for 24 h. After cooling, the mixture was concentrated in vacuo to give an oily residue. The residue was dissolved in dichloromethane and washed with brine, dried over anhydrous Na₂SO₄, filtered, and evaporated to give an oily crude product. The crude product was purified by silica gel column; elution with 20% EtOAc in hexane afforded the desired product YD0104 (methyl (1R,2R,3S,5S)-3-(benzoyloxy)-8-(5-(benzyloxy)-5-oxopentyl)-8-azabicyclo[3.2.1]octane-2-carboxylate) (337 mg, 73%) as an amorphous gel. ¹H NMR (300 MHz, CDCl₃) δ 8.09-8.01 (m, 2H), 7.54 (m, 1H), 7.49-7.29 (m, 7H), 5.26 (dt, J=12.0, 6.0 Hz, 1H), 5.14 (s, 2H), 3.68 (s, 4H), 3.32 (d, J=3.0 Hz, 1H), 3.09-2.98 (m, 1H), 2.53-2.34 (m, 3H), 2.25 (tt, J=12.0, 6.0 Hz, 2H), 2.14-1.93 (m, 2H), 1.91-1.82 (m, 1H), 1.79-1.64 (m, 4H), 1.52-1.35 (m, 2H). ¹³C NMR (75 MHz, CDCl₃) δ 173.48, 170.73, 166.19, 136.12, 132.91, 130.43, 129.72, 129.72, 128.56, 128.56, 128.33, 128.33, 128.23, 128.23, 128.19, 67.40, 66.10, 62.64, 60.51, 52.22, 51.24, 50.38, 35.71, 34.09, 28.43, 26.09, 25.50, 22.53.

A stirring solution of YD0104 (337 mg, 0.70 mmol) and 10% Pd/C (33.7 mg) in MeOH (10 mL) was evacuated and back-filled with H₂ at room temperature and one atmospheric pressure. After 12 h, the mixture was filtered over a pad of celite and the solvent was evaporated in vacuo. The resulting residue was further purified by silica gel column; elution with 10% MeOH in CH₂Cl₂ afforded the desired product YD0112 (205 mg, 75%) as a colorless amorphous gel. HPLC purity 98.0% (t_{R}=10.82 min). ¹H NMR (300 MHz, CD₃OD) δ 8.07-7.94 (m, 2H), 7.70-7.57 (m, 1H), 7.55-7.41 (m, 2H), 5.36 (dt, J=12.0, 6.2 Hz, 1H), 3.94 (m, 1H), 3.70 (s, 3H), 3.56 (m, 1H), 3.23 (dd, J=6.2, 3.1 Hz, 1H), 2.54 (t, J=7.2 Hz, 2H), 2.42 (td, J=12.0, 3.1 Hz, 1H), 2.22 (m, 4H), 2.03 (m, 1H), 1.88 (m, 2H), 1.75-1.48 (m, 4H). ¹³C NMR (75 MHz, CD₃OD) δ 184.81, 175.50, 169.72, 136.94, 133.80, 133.10, 133.10, 132.10, 132.10, 70.57, 65.68, 65.36, 55.94, 54.90, 52.80, 40.92, 38.49, 31.49, 28.68, 28.39, 27.27. HRMS Calcd for C₂₁H₂₈NO₆: [M+H]⁺ 390.1917; found 390.1920.

Scheme VII illustrating synthesis of 2-((1R,2R,3S,5S)-3-(benzoyloxy)-2-(methoxycarbonyl)-8-azabicyclo[3.2.1] octane-8-carbonyl)benzoic acid (YD0113).

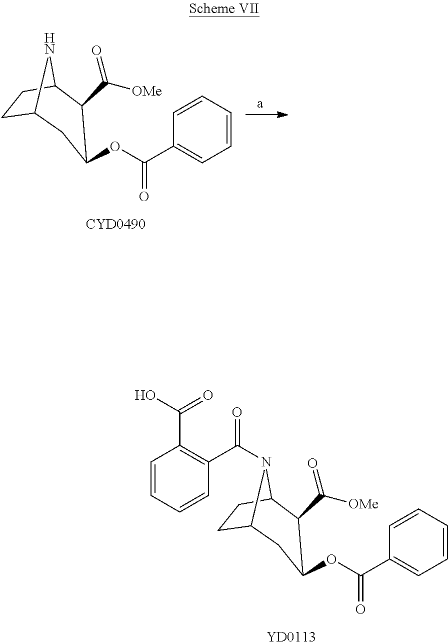 [SMILES: COC(=O)[C@@H]1C2CCC(C[C@@H]1OC(=O)c1ccccc1)N2 ; "CYD0490" ; COC(=O)[C@@H]1C2CCC(C[C@@H]1OC(=O)c1ccccc1)N2C(=O)c1ccccc1C(=O)O ; "YD0113"]

Reagents and conditions: (a) phthalic anhydride, Et₃N, CH₂Cl₂, rt, 12 h. To a solution of CYD0490 (459 mg, 1.58 mmol) and triethylamine (321 mg, 3.17 mmol) in dichloromethane (15 mL) was added phthalic anhydride (247 mg, 1.67 mmol) at room temperature. Then, the resulting mixture was stirred at room temperature for 12 h. The mixture was concentrated in vacuo to give an oily residue. The residue was dissolved in dichloromethane and washed with brine, dried over anhydrous Na₂SO₄, filtered, and evaporated to give an oily crude product. The crude product was purified by silica gel column; elution with 2.5% MeOH in dichloromethane afforded the desired product YD0113 (310 mg, 45%) as an amorphous gel. ¹H NMR (300 MHz, CDCl₃) δ 8.50 (m, 1H), 7.98 (m, 3H), 7.72-7.35 (m, 6H), 5.50 (dd, J=11.2, 5.7 Hz, 1H), 5.22 (s, 1H), 4.05-3.52 (m, 4H), 3.24 (m, 1H), 2.07 (m, 6H). ¹³C NMR (75 MHz, CD₂Cl₂) δ 168.44, 167.58, 163.82, 163.79, 131.35, 130.71, 127.83, 127.78, 127.76, 127.76, 127.05, 126.53, 126.51, 126.51, 126.48, 125.34, 64.74, 53.32, 50.33, 50.10, 46.78, 32.27, 26.00, 25.08.

Scheme VIII illustrating synthesis of methyl (1R,2R,3S,5S)-3-(benzoyloxy)-8-(2-mercaptoacetyl)-8-azabicyclo[3.2.1] octane-2-carboxylate (YD0322).

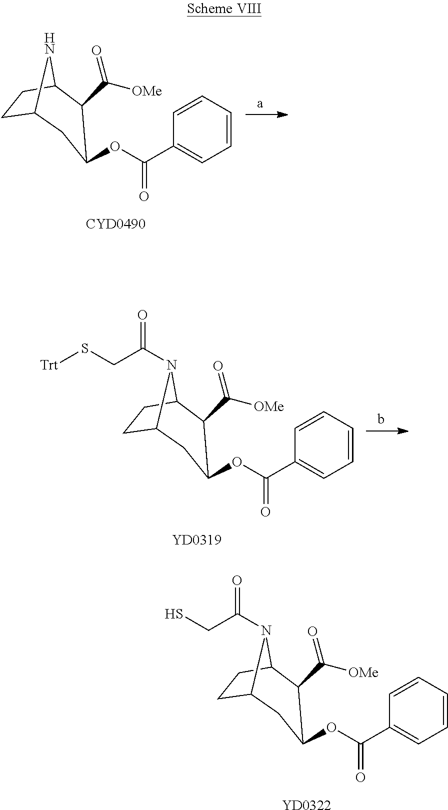

Reagents and conditions: (a) (tritylthio)acetic acid, HBTU, DIPEA, CH₂Cl₂, 0° C.→rt, 12 h; (b) TFA, Et₃SiH, CH₂Cl₂, rt, 2 h.

To a solution of CYD0490 (134 mg, 0.46 mmol) and (tritylthio)acetic acid (155 mg, 0.46 mmol) in dichloromethane (15 mL) was added DIPEA (180 mg, 1.39 mmol) and HBTU (264 mg, 0.70 mmol) at 0° C. The resulting mixture was stirred at room temperature for 12 h. The mixture was concentrated in vacuo to give a solid residue. The residue was dissolved in dichloromethane and washed with brine, dried over anhydrous Na₂SO₄, filtered, and evaporated to give crude product. The crude product was purified by silica gel column; elution with 25% EtOAc in hexanes afforded the desired product YD0319 (methyl (1R,2R,3S,5S)-3-(benzoyloxy)-8-(2-(tritylthio)acetyl)-8-azabicyclo[3.2.1]octane-2-carboxylate) (176 mg, 63%) as an white solid. ¹H NMR (300 MHz, CDCl₃) δ 7.94 (m, 2H), 7.60-7.38 (m, 9H), 7.37-7.19 (m, 9H), 5.51-5.32 (m, 1H), 4.98 (m, 1H), 3.68 and 3.39 (s, 3H), 3.13-2.97 (m, 2H), 2.32 (m, 1H), 2.03-1.65 (m, 6H). ¹³C NMR (75 MHz, CDCl₃) δ 169.93 and 169.78, 165.73 and 165.66, 164.73 and 164.42, 144.07 and 144.03 (3C), 133.26, 129.67 (2C), 129.58 (2C), 129.53(3 C), 129.51, 129.48 (3C), 128.42 (3C), 128.08 (3C), 126.89 (3C), 67.18 and 67.02 (1C), 66.49 and 66.31 (1C), 55.44 and 53.89 (1C), 53.00 and 52.04 (1C), 51.63 and 50.57 (1C), 48.78, 34.97 and 34.84 (1C), 34.46 and 33.21 (1C), 28.89 and 28.20 (1C), 27.04 and 26.53 (1C).

To a solution of YD0319 (90 mg, 0.15 mmol) in dichloromethane (7.5 mL) was added trifluoroacetic acid (1 mL) and Et₃SiH (19 mg, 0.16 mmol) at room temperature. The resulting mixture was stirred at room temperature for 2 h. The mixture was concentrated in vacuo to give an oily residue. The residue was dissolved in dichloromethane and washed with NaHCO₃ aqueous solution, brine, dried over anhydrous Na₂SO₄, filtered, and evaporated to give a solid crude product. The crude product was purified by silica gel column; elution with 33% EtOAc in hexanes afforded the desired product YD0322 (37 mg, 67%) as a white solid. ¹H NMR (300 MHz, CDCl₃) δ 8.04-7.90 (m, 2H), 7.64-7.53 (m, 1H), 7.50-7.39 (m, 2H), 5.65-5.43 (m, 1H), 5.14-4.85 (m, 1H), 4.64-4.31 (m, 1H), 3.68 (s, 3H), 3.41-3.21 (m, 2H), 3.21-3.12 (m, 1H), 2.61 (m, 1H), 2.34-1.78 (m, 6H). ¹³C NMR (75 MHz, CDCl₃) δ 170.32 and 170.00 (1C), 166.48 and 165.98 (1C), 165.75 and 165.70 (1C), 133.31, 129.68 (2C), 129.60, 128.44 (2C), 66.36 and 66.25 (1C), 55.99 and 54.23 (1C), 53.27 and 52.04 (1C), 51.96 and 50.98 (1C), 49.12 and 48.71 (1C), 34.41 and 33.47 (1C), 28.98 and 28.49 (1C), 26.96 and 26.53 (1C), 26.48 and 26.22 (1C).

Scheme IX illustrating synthesis of methyl (1R,2R,3S,5S)-3-(benzoyloxy)-8-(4-mercaptobutanoyl)-8-azabicyclo [3.2.1]octane-2-carboxylate (YD0326).

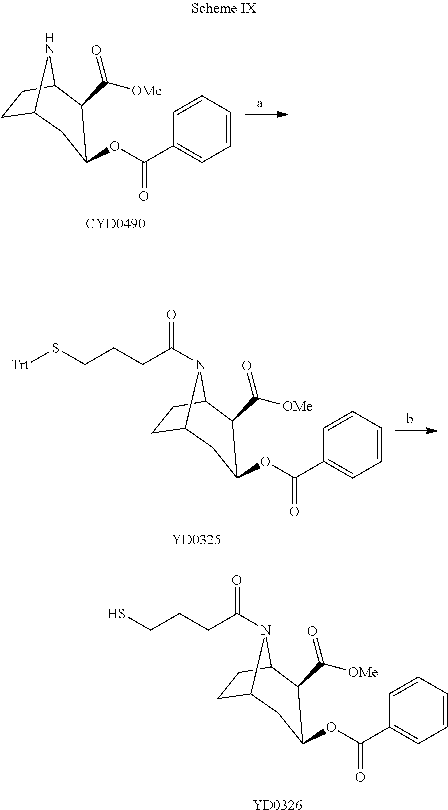

Reagents and conditions: (a) 4-[(triphenylmethyl)thio]-butanoic acid, HBTU, DIPEA, CH₂Cl₂, 0° C.→rt, 12 h; (b) TFA, Et₃SiH, CH₂Cl₂, rt, 2 h. To a solution of CYD0490 (84 mg, 0.29 mmol) and 4-[(triphenylmethyl)thio]-butanoic acid (105 mg, 0.29 mmol) in dichloromethane (15 mL) was added DIPEA (113 mg, 0.87 mmol) and HBTU (165 mg, 0.44 mmol) at 0° C. The resulting mixture was stirred at room temperature for 12 h. The mixture was concentrated in vacuo to give a solid residue. The residue was dissolved in dichloromethane and washed with brine, dried over anhydrous Na₂SO₄, filtered, and evaporated to give crude product. The crude product was purified by silica gel column; elution with 25% EtOAc in hexanes afforded the desired product YD0325 (methyl (1R,2R,3S,5S)-3-(benzoyloxy)-8-(4-(tritylthio)butanoyl)-8-azabicyclo[3.2.1]octane-2-carboxylate) (176 mg, 63%) as a white solid. ¹H NMR (300 MHz, CDCl₃) δ 7.98 (m, 2H), 7.66-7.51 (m, 1H), 7.49-7.39 (m, 8H), 7.35-7.29 (m, 4H), 7.28-7.18 (m, 5H), 5.62-5.36 (m, 1H), 5.12-4.83 (m, 1H), 4.50-4.22 (m, 1H), 3.62 (s, 3H), 3.15 (m, 1H), 2.60-2.09 (m, 5H), 2.05-1.66 (m, 7H). ¹³C NMR (75 MHz, CDCl₃) δ 170.02, 168.39, 165.76 and 165.72 (1C), 144.96 and 144.94 (3C), 133.24 (2C), 129.83, 129.68 (2C), 129.62 (6C), 128.42 (2C), 127.85 (6C), 126.60 (2C), 66.63 and 66.58 (1C), 66.41, 55.44 and 53.45 (1C), 52.77 and 51.92 (1C), 51.80 and 50.25 (1C), 49.41 and 48.76 (1C), 34.41 and 33.32 (1C), 32.67 and 32.19 (1C), 31.73 and 31.66 (1C), 29.18 and 28.59 (1C), 26.96 and 26.53 (1C), 24.30 and 24.23 (1C).

To a solution of YD0325 (117 mg, 0.15 mmol) in dichloromethane (7.5 mL) was added trifluoroacetic acid (1 mL) and Et₃SiH (24 mg, 0.20 mmol) at room temperature. The resulting mixture was stirred at room temperature for 2 h. The mixture was concentrated in vacuo to give an oily residue. The residue was dissolved in dichloromethane and washed with NaHCO₃ aqueous solution, brine, dried over anhydrous Na₂SO₄, filtered, and evaporated to give a solid crude product. The crude product was purified by silica gel column; elution with 50% EtOAc in hexanes afforded the desired product YD0326 (67 mg, 91%) as a white solid. ¹H NMR (300 MHz, CDCl₃) δ 8.04-7.90 (m, 2H), 7.57 (m, 1H), 7.50-7.37 (m, 2H), 5.63-5.43 (m, 1H), 5.03 (m, 1H), 4.63-4.33 (m, 1H), 3.68 (s, 3H), 3.18 (m, 1H), 2.73-2.42 (m, 4H), 2.40-1.73 (m, 8H). ¹³C NMR (75 MHz, CDCl₃) δ 170.10, 168.27, 165.76, 133.24, 129.68 (2C), 129.61, 128.41 (2C), 66.54 and 66.39 (1C), 55.42 and 53.49 (1C), 52.85 and 51.85 (1C), 51.92 and 50.28 (1C), 49.37 and 48.72 (1C), 34.38 and 33.36 (1C), 31.46 and 31.14 (1C), 29.26 and 29.07 (1C), 28.95 and 28.65 (1C), 26.95 and 26.53 (1C), 24.31 and 24.21 (1C).

Scheme X illustrates synthesis of methyl (1R,2R,3S,5S)-3-(benzoyloxy)-8-(prop-2-yn-1-yl)-8-azabicyclo[3.2.1] octane-2-carboxylate (YD0334).

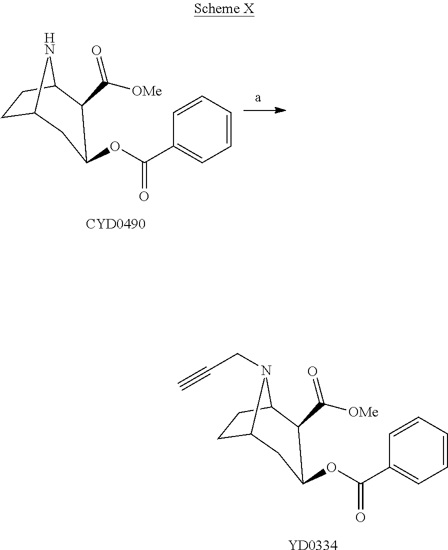 [SMILES: COC(=O)[C@@H]1C2CCC(C[C@@H]1OC(=O)c1ccccc1)N2 ; "CYD0490" ; C#CCN1C2CCC1[C@@H](C(=O)OC)[C@@H](OC(=O)c1ccccc1)C2 ; "YD0334"]

Reagents and conditions: (a) 3-bromopropyne, K₂CO₃, MeCN, reflux, 24 h. A mixture of CYD0490 (352 mg, 1.22 mmol), propargyl bromide (174 mg, 1.46 mmol), and K₂CO₃ (673 mg, 4.87 mmol) was refluxed in acetonitrile (15 mL) for 24 h. The solvent was removed, and the residue was dissolved in ice-cold ammonia solution (2% v/v). The aqueous solution was extracted with dichloromethane, washed with NaHCO₃ aqueous solution, brine, dried over anhydrous Na₂SO₄, filtered, and evaporated to give an oily residue. The residue was purified using silica gel column; elution with 20% EtOAc in hexanes afforded the desired product YD0334 as a colorless amorphous gel (312 mg, 78%). ¹H NMR (300 MHz, CDCl₃) δ 8.08-8.01 (m, 2H), 7.59-7.50 (m, 1H), 7.47-7.38 (m, 2H), 5.27 (m, 1H), 4.03-3.87 (m, 1H), 3.73 (s, 3H), 3.52-3.39 (m, 1H), 3.28-3.03 (m, 3H), 2.50 (m, 1H), 2.19 (m, 1H), 2.17-1.68 (m, 5H). ¹³C NMR (75 MHz, CDCl₃) δ 170.31, 166.19, 132.95, 130.30, 129.74 (2C), 128.33 (C), 81.02, 71.33, 67.02, 62.06, 60.19, 51.39, 50.16, 41.95, 35.58, 25.99, 25.35.

Scheme XI illustrates synthesis of methyl (1R,2R,3S,5S)-3-(benzoyloxy)-8-(5-(2,5-dioxo-2,5-dihydro-1H-pyrrol-1-yl)pentanoyl)-8-azabicyclo[3.2.1]octane-2-carboxylate (YD0542).

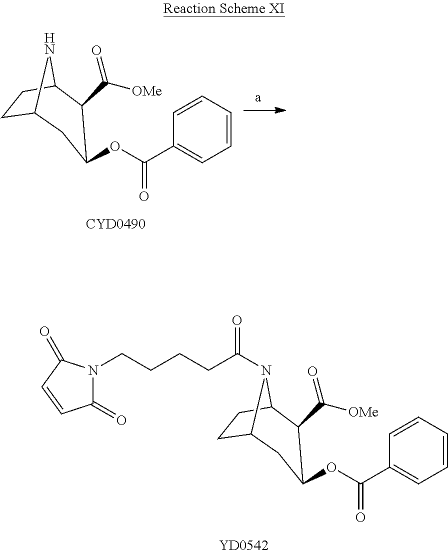 [SMILES: COC(=O)[C@@H]1C2CCC(C[C@@H]1OC(=O)c1ccccc1)N2 ; "CYD0490" ; COC(=O)[C@@H]1C2CCC(C[C@@H]1OC(=O)c1ccccc1)N2C(=O)CCCCN1C(=O)C=CC1=O ; "YD0542"]

Reagents and conditions: (a) 5-maleimidopentanoic acid, EDCI, DMAP, CH₂Cl₂, rt, 12 h. To a solution of CYD0490 (112 mg, 0.39 mmol) and 5-maleimidopentanoic acid (77 mg, 0.39 mmol) in dichloromethane (10 mL) was added DMAP (10 mg, 0.08 mmol) and EDCI (112 mg, 0.59 mmol) at room temperature. The resulting mixture was stirred at room temperature for 12 h. The mixture was concentrated in vacuo to give an oily residue. The residue was dissolved in dichloromethane and washed with NaHCO₃ aqueous solution, brine, dried over anhydrous Na₂SO₄, filtered, and evaporated to give a solid crude product. The crude product was purified by silica gel column; elution with 2% MeOH in dichloromethane afforded the desired product YD0542 (76 mg, 42%) as amorphous gel. ¹H NMR (300 MHz, CDCl₃) δ 8.01-7.91 (m, 2H), 7.56 (m, 1H), 7.43 (m, 2H), 5.50 (m, 1H), 4.99 (m, 1H), 4.57-4.29 (m, 1H), 3.84-3.30 (m, 6H), 3.26-3.04 (m, 1H), 2.64-2.24 (m, 3H), 2.21-1.79 (m, 6H), 1.68 (d, J=4.8 Hz, 4H). ¹³C NMR (75 MHz, CDCl₃) δ 173.49, 170.21 and 170.09 (1C), 169.16, 168.41, 165.76, 133.25, 129.76, 129.67 (2C), 129.60, 129.28, 128.43 (2C), 66.53 and 66.39 (1C), 55.45 and 53.47 (1C), 52.83 and 51.80 (1C), 51.95 and 50.27 (1C), 49.37 and 48.69 (1C), 38.62 and 34.36 (1C), 34.28 and 33.36 (1C), 32.61 and 32.28 (1C), 29.26 and 28.65 (1C), 27.28, 26.93 and 26.51 (1C), 22.21 and 22.05 (1C).

Scheme XII illustrates synthesis of 5-((1R,2R,3S,5S)-3-((4-fluorobenzoyl)oxy)-2-(methoxycarbonyl)-8-azabicyclo [3.2.1]octan-8-yl)-5-oxopentanoic acid (PV0104).

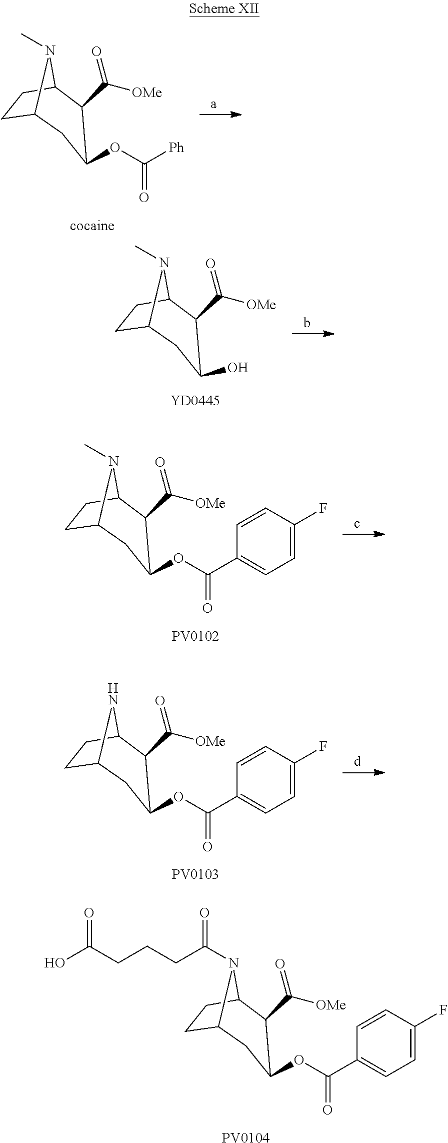

Reagents and conditions: (a) H₂SO₄, MeOH, H₂O, reflux, 12 h; (b) 4-fluorobenzoyl chloride, Et₃N, CH₂Cl₂, rt, 12 h; (c) 1-chloroethyl chloroformate, CH₂C1CH₂C1, 90° C., Na₂CO₃, 12 h; MeOH, reflux, 1 h; (d) glutaric anhydride, DMAP, DIPEA, CH₂Cl₂, 45° C. Synthesis of the key intermediates YD0445 and PV0102 were followed the reported procedures (J. Am. Chem. Soc. 2013, 135, 2971-2974).

To a solution of PV0102 (179.9 mg, 0.560 mmol) in dichloroethane (10 mL) was added 1-chloroethyl chloroformate (0.30 mL, 2.80 mmol) and sodium carbonate (296.7 mg, 2.80 mmol) at room temperature. The resulting mixture was reflux for overnight. Then the mixture was filtered and concentrated in vacuo to give an oily intermediate. The intermediate was refluxed in MeOH (10 mL) for 1 h. The solvent was removed, and the residue was diluted with H₂O, basified (pH=8-9) with NaHCO₃ aqueous solution, extracted with dichloromethane. The organic layer was washed with brine, dried with Na₂SO₄, filtered, evaporated to give an oily crude product (156 mg, 90%). The crude product was directly used in the next step reaction without further purification. ¹H NMR (300 MHz, CDCl₃) δ 7.98-7.92 (m, 2H), 7.12-7.04 (m, 2H), 5.41 (m, 1H), 3.94 (m, 1H), 3.80 (m, 2H), 3.63 (s, 3H), 3.12-3.05 (m, 1H), 2.20-1.92 (m, 4H), 1.79-1.66 (m, 2H). ¹³C NMR (75 MHz, CDCl₃) δ 172.54 and 167.51 (1C), 164.49 and 164.14 (1C), 132.11, 131.98, 126.10, 126.05, 115.70, 115.41, 66.87, 55.89, 53.18, 51.78, 48.01, 34.78, 28.03, 27.07.

To a solution of PV0103 (methyl (1R,2R,3S,5S)-3-((4-fluorobenzoyl)oxy)-8-azabicyclo[3.2.1]octane-2-carboxylate) (89.5 mg, 0.291 mmol) and DIPEA (75.2 mg, 0.582 mmol) in DCM (10 mL) was added DMAP (3.66 mg, 0.0300 mmol) and glutaric anhydride (33.25 mg, 0.291 mmol) at room temperature. The reaction was stirred at 45° C. for 18 h. The reaction mixture was then diluted with water and extracted with dichloromethane. The extract was washed with brine and dried over anhydrous Na₂SO₄, filtered, and evaporated to give an oily residue. The residue was purified using silica gel column; elution with 10% MeOH in CH₂Cl₂ afforded the desired product PV0104 as a colorless amorphous gel (73 mg, 60%). ¹H NMR (300 MHz, CDCl₃) δ 7.98 (m, 2H), 7.10 (m, 2H), 5.50 (m, 1H), 5.16-4.89 (m, 1H), 4.60-4.31 (m, 1H), 3.67 (s, 3H), 3.28-3.07 (m, 1H), 2.65-2.38 (m, 4H), 2.38-1.69 (m, 8H). ¹³C NMR (75 MHz, CDCl₃) δ 177.43 and 170.12 (1C), 168.90 and 168.81 (1C), 167.62 and 164.76 (1C), 164.70 and 164.24 (1C), 132.31, 132.23 and 132.11 (1C), 132.19, 125.93 and 125.89 and 125.85 (1C), 115.78 and 115.75 (1C), 115.49 and 115.46 (1C), 66.65 and 66.45 (1C), 55.41 and 53.61 (1C), 53.02 and 52.04 (1C), 51.83 and 50.41 (1C), 49.25 and 48.67 (1C), 34.38 and 33.31 (1C), 33.25 and 32.38 (1C), 31.98 and 29.69 (1C), 29.10 and 28.52 (1C), 26.86 and 26.49 (1C), 20.20 and 20.11 (1C). HRMS Calcd for C₂₁H₂₅FNO₇: [M+H]⁺ 422.1615; found 422.1599.

Scheme XIII illustrates synthesis of 5-((1R,2R,3S,5S)-3-((4-fluorobenzoyl)oxy)-2-(methoxycarbonyl)-8-azabicyclo [3.2.1]octan-8-yl)pentanoic acid (PV0106).

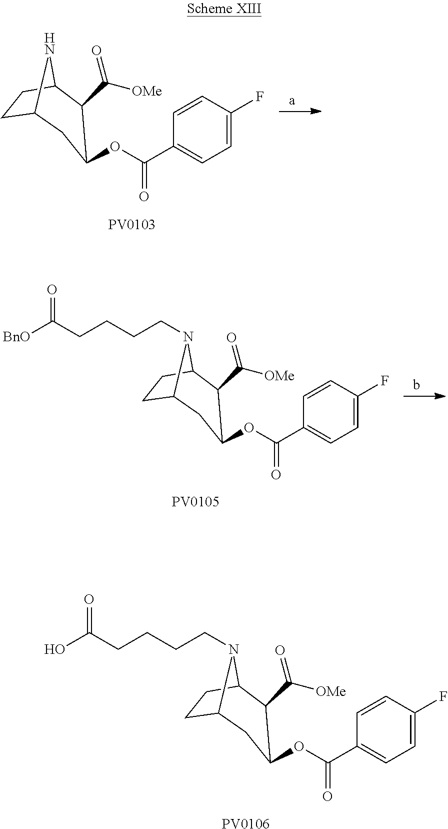 [SMILES: "PV0103" ; COC(=O)[C@@H]1C2CCC(C[C@@H]1OC(=O)c1ccc(F)cc1)N2 ; "PV0105" ; COC(=O)[C@@H]1C2CCC(C[C@@H]1OC(=O)c1ccc(F)cc1)N2CCCCC(=O)OCc1ccccc1 ; "PV0106" ; COC(=O)[C@@H]1C2CCC(C[C@@H]1OC(=O)c1ccc(F)cc1)N2CCCCC(=O)O]

Reagents and conditions: (a) BnOCOCH₂CH₂CH₂CH₂Br, K₂CO₃, KI, acetone, 12 h; (e) H₂, Pd-C, MeOH, rt, overnight. Compound PV0105 (methyl (1R,2R,3S,5S)-8-(5-(benzyloxy)-5-oxopentyl)-3-((4-fluorobenzoyl)oxy)-8-azabicyclo[3.2.1]octane-2-carboxylate) (416.6 mg) was prepared in 56% yield by a procedure similar to that used to prepare compound YD0104. The title compound was obtained as a yellowish gel. ¹H NMR (300 MHz, CDCl₃) δ 8.13-7.97 (m, 2H), 7.48-7.30 (m, 5H), 7.16-7.04 (m, 2H), 5.29-5.17 (m, 1H), 5.13 (s, 2H), 3.68 (m, 4H), 3.32 (m, 1H), 3.08-2.98 (m, 1H), 2.48-2.33 (m, 3H), 2.32-2.16 (m, 2H), 2.12-1.91 (m, 2H), 1.91-1.80 (m, 1H), 1.71 (m, 4H), 1.41 (m, 2H). ¹³C NMR (75 MHz, CDCl₃) δ 173.44, 170.63 and 167.42 (1C), 165.19 and 164.06 (1C), 136.08, 132.31, 132.18, 128.53 (2C), 128.21 (2C), 128.17, 126.66, 126.63, 115.58, 115.29, 67.56, 66.10, 62.59, 60.49, 52.21, 51.25, 50.31, 35.69, 34.07, 28.41, 26.08, 25.46, 22.52.

Compound PV0106 (93 mg) was prepared in 64% yield by a procedure similar to that used to prepare compound YD0112. The title compound was obtained as a yellowish gel. ¹H NMR (300 MHz, CDCl₃) δ 8.15-7.88 (m, 2H), 7.05 (m, 2H), 5.23 (m, 1H), 3.84-3.72 (m, 1H), 3.40 (s, 4H), 3.15-3.00 (m, 1H), 2.56-2.19 (m, 5H), 2.20-1.95 (m, 2H), 1.69 (m, 4H), 1.47 (m, 2H), 1.22 (m, 1H). ¹³C NMR (75 MHz, CDCl₃) δ 178.00, 170.88 and 167.46 (1C), 165.07 and 164.09 (1C), 132.27, 132.15, 126.30, 126.26, 115.62, 115.32, 66.97, 61.92, 60.87, 51.87, 51.61, 50.21 and 49.50 (1C), 35.06, 34.38, 27.64, 25.57, 25.28, 22.24. HRMS Calcd for C₂₁H₂₇FNO₆: [M+H]⁺ 408.1822; found 408.1809.

### II. Synthesis of Self-Assembling Peptide-Cocaine Conjugates

Cocaine haptens were coupled to self-assembling peptides for use as immunogens for antibody production against small drug molecules. In one embodiment, analogs as described above can be coupled with peptide KFE8 (amino acid sequence FKFEFKFE (SEQ ID NO:1)) through a triple glycine spacer at the peptide's N-terminus (GGG-KFE8).

Peptide nanofiber carriers are attractive as antigen delivery scaffolds due to their inert and biocompatible nature (Matson et al., 2012). Synthetic peptides disclosed herein can be easily produced through standard peptide synthesis protocols and peptides incorporating non-natural amino acids or non-peptidic moieties can be designed to reduce proteolysis and degradation of the vaccine in vivo. The chemical versatility of the self-assembling peptide platform disclosed herein allows for conjugation of TLR agonists and/or CD4 T helper epitopes during synthesis for enhanced immunogenicity and antibody production. In addition, physical conjugation of TLR agonists will minimize their free dissemination into the surrounding tissue and reduce local inflammation.

Generally, the peptides disclosed herein may be synthesized, functionalized, and purified by means known by one of skill in the art. A non-limiting example of a means of synthesizing a peptide include synthesized on a CSBio136-XT peptide synthesizer using standard Fmoc chemistry. A non-limiting example of a purification means include purification using reverse-phase HPLC and water/acetonitrile gradients. Peptide identity and purity can be determined by means known by one of skill in the art, as an non-limiting example, MALDI-MS may be used.

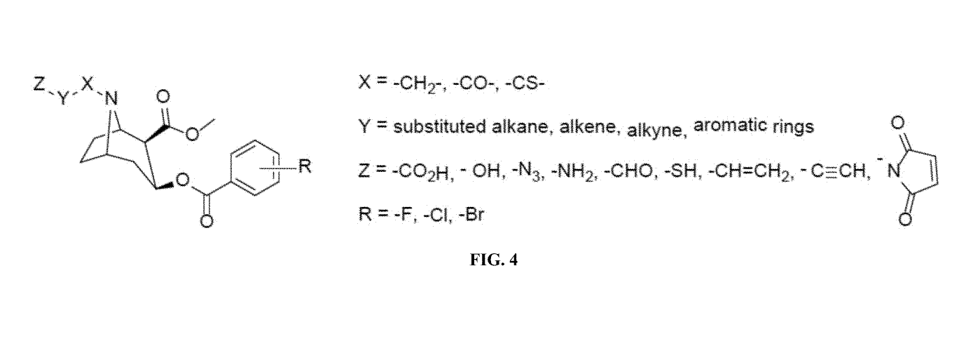

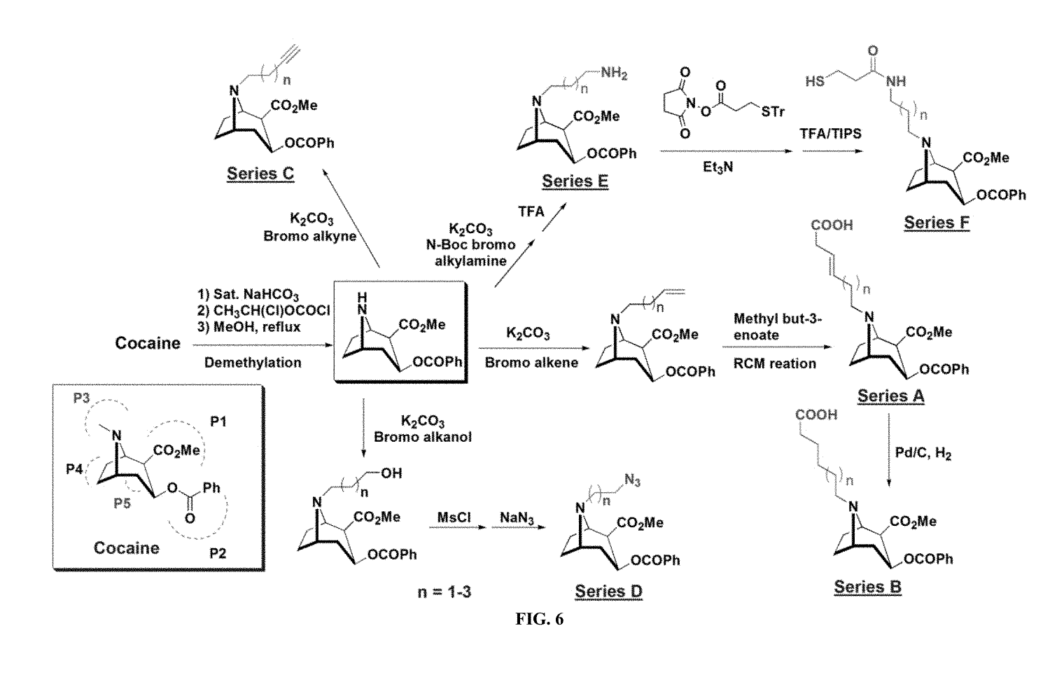

As a non-limiting example, the peptides can be functionalized with chemical groups orthogonal to modifications made to cocaine to produce the cocaine haptens. For example, an amine group on the peptide may be used to bind a carboxyl group added to the P3 moiety of cocaine. Other combinations may include chemical modification using succinimidyl esters, maleimide, azide, and alkyne groups on the peptide. Some non-limiting examples include those orthogonal chemical groups included in FIG. 7. Self-assembling peptide-cocaine conjugates disclosed herein include, but are not limited to, those shown by Formula II (FIG. 5):

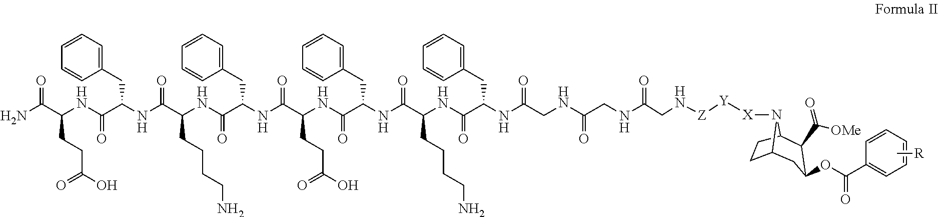

wherein
X is —CH₂—, —CO—, or —CS—;
Y is substituted alkane, alkene, alkyne, or aromatic rings;
Z is —CO—, —CH₂CONH—, —COCH₂CONH—, —CH₂OCH₂—, —CH₂—, —COCH₂SS,

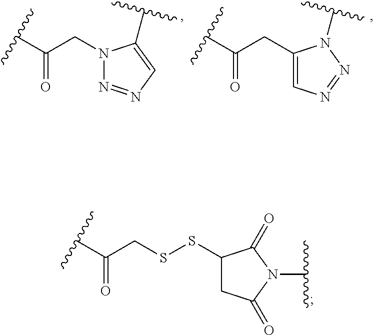 [SMILES: *C(=O)Cn1nncc1* ; *C(=O)Cc1cnnn1* ; *C(=O)CSSC1CC(=O)N(*)C1=O]

and
and R is hydrogen, hydroxyl, nitro, mercapto, cyano, azido, alkyl, heteroalkyl, alkoxy, halogen, amino, oxo, or alkylsulfonyl. In certain aspects R is in particular —F, —Cl, or —Br. In certain aspects R is at the 2, 3, 4, 5, or 6 position.

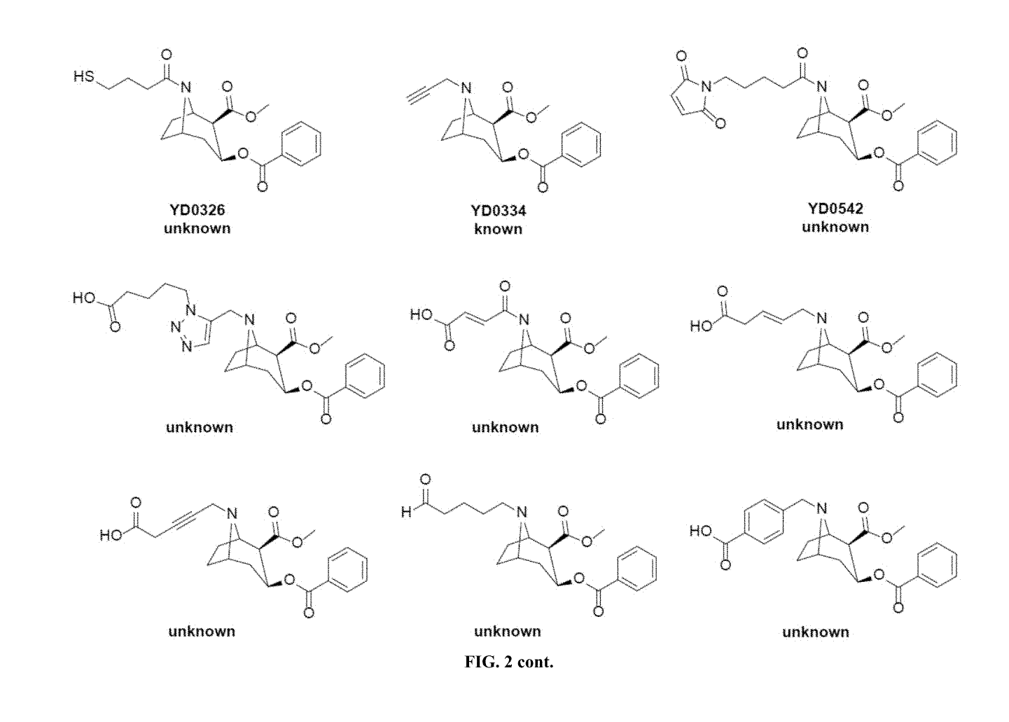

In some embodiments, the self-assembling peptide-cocaine conjugates include those described in FIG. 3. In some embodiments, the self-assembling peptide-cocaine conjugates use the cocaine haptens described by Formula I or in FIGS. 2, 4, 6, 8, and 9. Synthesis of a non-limiting example of a self-assembling peptide-cocaine conjugate is described in detail below.

Peptide KFE8 (FKFEFKFE or NH₂-GGGFKFEFKFE-CONH) with a triple glycine spacer at its N-terminus (GGG-KFE8) (5 of FIG. 10) was synthesized using solid phase Fmoc chemistry on a CS Bio-CS336X solid phase peptide synthesizer (CS Bio, CA). Rink Amide MBHA (Novabiochem, MA) was swelled in dry DMF (dimethylformamide) for 1 hr, and amino acids were coupled using HBTU (O-(Benzotriazol-1-yl)-N,N,N′,N′-tetramethyluronium hexafluorophosphate)/DIEA (diisopropylethylamine) for 30 min followed by HOBt (1-Hydroxybenzotriazole)/DIC (diisopropylcarbodimide) for 2 h. The N-terminal Fmoc was removed using 20% piperidine to yield a primary amine (6 of FIG. 10). YD0112 was then coupled with 6 on resin using HBTU/DIEA with stirring for 2 hours or overnight to yield cocaine-KFE8 conjugate (7 of FIG. 10). The conjugate was cleaved from the resin using 95% TFA/2.5% H2O/2.5% triisopropyl silane cocktail and washed 3 times in diethyl ether to yield the immunogen, CocKFE8 (8 of FIG. 10). CocKFE8 was purified using reverse-phase HPLC using water acetonitrile gradients on a C18 column to >90% purity. Peptide mass was confirmed by MALDI using α-cyano-4-hydroxycinnamic acid matrix (Bruker Daltonics, MA). Molecular weight of CocKFE8 was verified using a Bruker MALDI-TOF mass spectrometer calc'd: [M+H]⁺1662.54; found 1663.16. Peptide was lyophilized and stored at 4° C. and endotoxin levels were tested using a limulus amebocyte lystae (LAL) chromogenic end point assay (Lonza, MA) at the same volume and concentration used for immunizations and were found to be less than 0.1 EU/mL and within acceptable limits.

To determine the assembly and secondary structure characteristics of CocKFE8 the conjugate was dissolved in pure water (1 mM), incubated overnight at room temperature or 4° C., and diluted in PBS (0.33 mM) to induce fibrillization for 4 h at room temperature. Control fibers of peptide KFE8 were prepared in similar fashion and aggregates were analyzed by transmission electron microscopy. Briefly, the fibers were applied to 300 mesh copper grids with carbon support film (Quantifoil). The grids were negatively stained with 2% uranyl acetate, and imaged on a JEM1400 TEM (JEOL) equipped with LaB₆ electron gun and digital cameras. Images were viewed and recorded with an Ultrascan 1000 camera (Gatan).

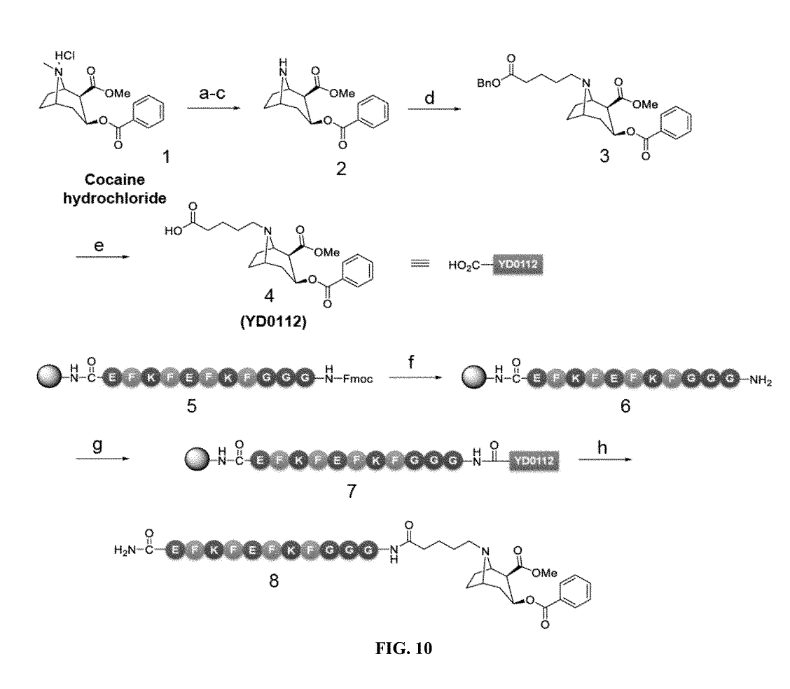

Results indicated that the CocKFE8 conjugate assembled into nanofibers (FIG. 11B) similar to KFE8 with fibrillar morphology (FIG. 11A) suggesting that the presence of cocaine did not inhibit the self-assembly process significantly.

The secondary structure of CocKFE8 nanofibers compared to KFE8 nanofibers was investigated using circular dichrosim spectroscopy. Briefly, CD experiments were carried out on a JASCO J-815 CD Spectrometer. Peptide stock solutions (1 mM) were made in ultra-pure water and diluted to working concentrations before use. The CD wavelength range was from 300 nm to 195 nm with a scanning speed of 0.3 nm/s and a bandwidth of 0.5 nm. CD spectra were recorded at room temperature with a fixed-path-length (1 mm) cell. The solvent background contribution was subtracted.

Spectra for peptide KFE8 showed signals at ˜218 nm and ˜205 nm indicative of β-sheet secondary structure and π-π effects of Phe aromatic groups respectively. Spectra of CocKFE8 conjugate showed a loss of signal at 205 nm suggesting weakened π-π stacking (FIG. 11C). Nonetheless, this did not significantly affect the ability of the conjugate to self-assemble into nanofiber scaffolds, which is crucial for eliciting adjuvant-free immune responses (Rudra et al., Acs Nano, 2012).

### III. Immunogenicity, Dose Dependence, and Route of CocKFE8 Delivery

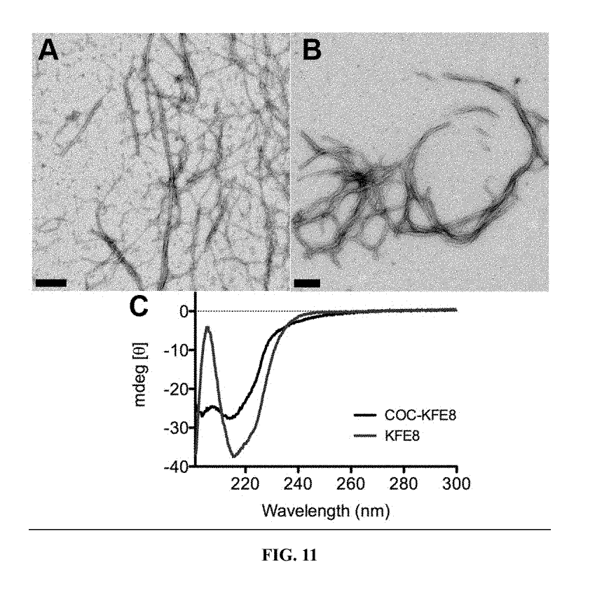

To assess immunogenicity, dose dependence, and route of CocKFE8 delivery, C57BL6 mice were vaccinated either subcutaneously (SC) or intraperitoneally (IP) with three doses of CocKFE8 nanofibers (10, 50, and 100 μg) and boosted with the same dose at 21, 42, and 63 days (timeline, FIG. 12). As a control, a commercially available cocaine-BSA conjugate (50 μg via SC and IP routes) was included.

Animals and Immunizations Methodology—

Male mice (C57BL/6, 5-6 weeks old) purchased from Jackson labs (State, USA). Mice were allowed to acclimate for 5-7 days in a colony room at a constant temperature (21-23° C.) and humidity (45-50%) on a 12 hr light-dark cycle (lights on 0700-1900 hr). Mice were housed five per cage with food and water available ad libitum throughout all phases of the studies. For vaccination, the stock solution of Cocaine-KFE8 (CocKFE8) peptide nanofibers was prepared freshly each time. First the peptide was dissolved in water (8 mM stock), incubated overnight, and diluted 4-fold in PBS (2 mM stock) 4 h prior to vaccination. At the injected concentration, CocKFE8 solution was viscous gel and could be easily manipulated using a 25-gauge syringe. Mice were vaccinated IP or SC route with 50 μl of CocKFE8 nanofibers and boosted with the same dosage at days 21, 42, and 63. Cocaine-BSA conjugate was purchased from Fritzgerald Industries (Acton, Mass.) and used without further modification. Blood was collected via the submandibular vein two weeks after the last boost and sera stored at −80° C. All experiments were repeated independently twice with 10-15 mice per group per experiment.

ELISA data indicated that 50 μg of CocKFE8 nanofibers delivered via the IP route elicited a better antibody response, as assessed by ELISA, compared to other routes and doses (data not shown). Delivery of CocKFE8 nanofibers via the SC route failed to elicit any antibodies.

ELISA Methodology—

Briefly, high-binding ELISA plates (eBioscience, CA, USA) were coated with 20 μg/mL of CocKFE8 nanofibers in PBS overnight at 4° C. and blocked with 200 μL of 1% BSA in PBST (0.5% Tween-20 in PBS) for 1 h. Serum was diluted in PBST (1:100) and applied (100 μL/well) for 1 h at room temperature followed by peroxidase-conjugated goat anti-mouse IgG (H+L) (Jackson Immuno Research, PA, USA) (1:5000 in 1% BSA-PBST, 100 μL/well). Plates were developed using TMB substrate (100 μL/well, eBioscience, CA, USA), the reaction stopped using 50 μl of 1 M phosphoric acid, and absorbance measured at 450 nm. Absorbance values of PBS (no antigen)-coated wells were subtracted to account for background. Vaccinated mice with titers lower than three times the standard deviation of the mean titer for the control group (PBS-treated; titers lower than 0.05 absorbance units) were defined as “CocKFE8 non-responders” and all other vaccinated mice were defined as “CocKFE8 responders” and compared to control mice in the behavioral studies.

In previous investigations, nanofiber vaccines using protein and peptide antigens have been immunogenic by both SC and IP routes. Both of these routes are routinely used in rodents to assess vaccine efficacy. Not to be bound by theory, our hypothesis is that the vaccine administered IP led to the recruitment of peritoneal macrophages and efficient processing by antigen-presenting cells as reported previously (Chen et al., 2013). It is possible that administration of the vaccine via the SC route with a depot-forming adjuvant would lead to stronger antibody responses. Furthermore, cocaine-BSA conjugates delivered through IP and SC routes failed to induce anti-cocaine antibodies suggesting that cocaine-carrier conjugates are ineffective at inducing antibody responses without added adjuvants (data not shown).

### IV. Antibody Functionality and Suppression of Cocaine-Evoked Hyperactivity In Vivo

To assess antibody functionality and suppression of cocaine-evoked hyperactivity in vivo, mice were vaccinated with 50 μg of CocKFE8 nanofibers IP and boosted according schedule shown in FIG. 12. Control mice were injected with phosphate-buffered saline (PBS). The sera were analyzed using ELISA as described above and data indicated that CocKFE8 nanofibers induced anti-cocaine antibody responses without the need for exogenous adjuvants (CocKFE8 vs. control, p<0.05; FIG. 12A). A broad distribution in antibody levels was observed and to assess functionality and specificity for cocaine in behavioral assessments, vaccinated mice were divided into two groups. Mice with absorbance values lower than the control (PBS-treated) group mean plus three times the standard deviation (i.e., <0.05, represented by the dashed line, FIG. 12A) were defined as “CocKFE8 non-responders” (FIG. 12A). All other vaccinated mice with significantly higher absorbance values (p<0.05 vs. control and CocKFE8 non-responders, FIG. 12A) were defined as “CocKFE8 responders”. The number of non-responders was approximately 30%, which is similar to what has been observed using cocaine-carrier conjugate vaccines in clinical trials (˜67% responders) (Kosten et al., Drug and alcohol dependence, 2014). This attests to the fact that small molecule drugs are poorly immunogenic and that principles of vaccine development applied to infectious diseases cannot be directly translated to addiction vaccines. The non-responders were separated and treated as an independent cohort for behavior analyses since they were immunologically different from the controls and displayed significantly lower antibody levels relative to responders (CocKFE8 non-responders vs. CocKFE8 responders, p<0.05; FIG. 12A).

Cocaine-evoked locomotor activity was measured two weeks after the last boost (Valencia et al., 2012; Yan et al., 2013). Mice were removed from their home cages and placed in activity monitors for 60 min to assess spontaneous motor activity in vaccinated and control groups. Following the assessment of spontaneous motor activity, mice were injected with cocaine (15 mg/kg, IP) and evaluated for cocaine-evoked locomotor activity for 60 min.

Locomotor Activity Methodology—

Two weeks after the last booster dose, blood collection was performed in vaccinated and PBS-treated control mice for antibody detection using ELISA. The following day, spontaneous motor activity and cocaine-evoked locomotor activity were assessed in control and vaccinated mice under low light conditions using an open field activity system (San Diego Instruments, San Diego, Calif., USA) according to a modified protocol from previous publications (Yan et al., 2013; Orson et al., 2013). Clear Plexiglas chambers (40×40×40 cm³) were surrounded by two 4×4 photobeam arrays. The lower array, positioned level with the chamber floor recorded horizontal activity. Peripheral activity was defined as the sum of photobeam breaks that occurred within the outer 12 cm band of the activity monitor. For all experiments, spontaneous motor activity was recorded in the activity monitors for the first 60 minutes. Immediately following the assessment of spontaneous motor activity, mice were injected with cocaine (15 mg/kg, 10 ml/kg; IP) and returned to the activity monitors for an additional hour to assess cocaine-evoked locomotor activity.

Statistical Analysis—

Spontaneous and cocaine-evoked locomotor activity data were analyzed and are presented as mean total peripheral activity summed across the 60-min session for control, CocKFE8 responder and CocKFE8 non-responder groups, respectively (FIGS. 12B, 12C, and not shown). Differences in the mean total peripheral activity (spontaneous and cocaine-evoked) observed for the vaccinated mice (CocKFE8 responders and cocKFE8 non-responders) relative to controls were analyzed using one-tailed Student's t-test. All statistical analyses were conducted with an experiment-wise error rate of α=0.05. The antibody titers (absorbance measured using ELISA) and cocaine-evoked peripheral activity (counts/60 min) measures for individual mice were transformed by square root transformations to confirm normality of distribution. Transformed data were employed in Pearson's correlation analysis (SAS, version 9.4; Cary, N.C., USA).

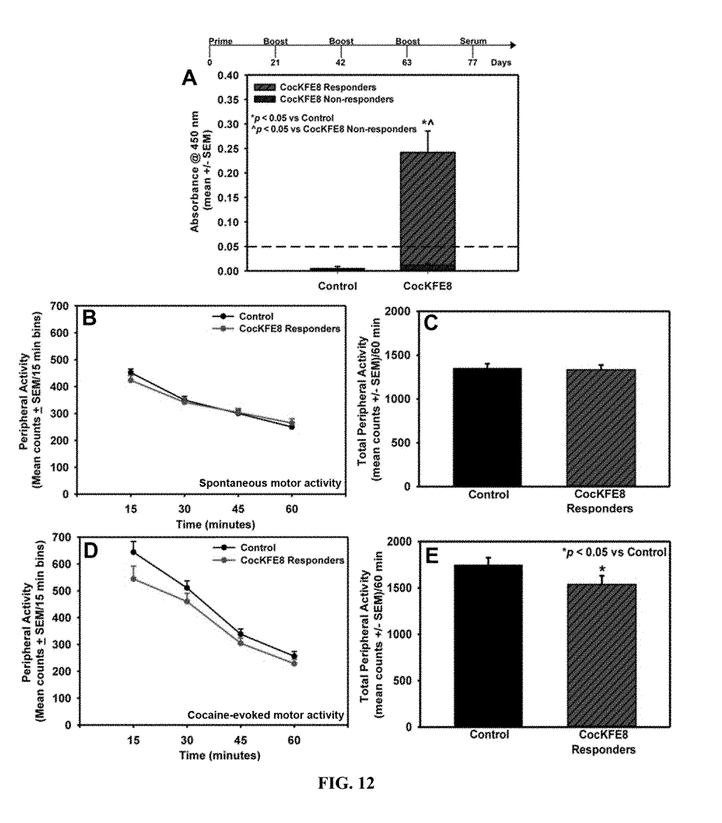

No significant differences in spontaneous motor activity was observed between control and vaccinated mice (p>0.05, n.s.; FIG. 12B). The effect of vaccine in suppressing cocaine-evoked hyperactivity was apparent in the first 15 min time bin following cocaine injection (Control vs. CocKFE8-responders, p=0.056). Analysis of the total cocaine-evoked peripheral activity measured across the entire 60-min test session in vaccinated mice defined as CocKFE8 responders compared to control mice, indicated a modest yet significant decrease (p<0.05; FIG. 12C). To confirm antibody-dependence of the reduced response to cocaine, spontaneous and cocaine-evoked activity was measured in CocKFE8 non-responder relative to control mice. Spontaneous and cocaine-evoked locomotor activity in non-responder mice did not differ relative to control mice (p>0.05, n.s.; data not shown). Furthermore, a significant negative correlation was observed between antibody levels and cocaine-evoked hyperactivity for individual vaccinated mice suggesting that CocKFE8 nanofibers induced cocaine-specific antibodies (FIG. 13).

### REFERENCES

Center for Behavioral Health Statistics and Quality. (2015). Behavioral health trends in the United States: Results from the 2014 National Survey on Drug Use and Health (HHS Publication No. SMA 15-4927, NSDUH Series H-50).
Aguilar, J. C.; Rodriguez, E. G. Vaccine adjuvants revisited. Vaccine 2007, 25, 3752.
Alving, C. R.; Matyas, G. R.; Tones, O.; Jalah, R.; Beck, Z. Adjuvants for vaccines to drugs of abuse and addiction. Vaccine 2014, 32, 5382.
Chen, J. J.; Pompano, R. R.; Santiago, F. W.; Maillat, L.; Sciammas, R.; Sun, T.; Han, H. F.; Topham, D. J.; Chong, A. S.; Collier, J. H. The use of self-adjuvanting nanofiber vaccines to elicit high-affinity B cell responses to peptide antigens without inflammation. Biomaterials 2013, 34, 8776.
Chesson, C. B.; Huelsmann, E. J.; Lacek, A. T.; Kohlhapp, F. J.; Webb, M. F.; Nabatiyan, A.; Zloza, A.; Rudra, J. S. Antigenic peptide nanofibers elicit adjuvant-free CD8(+) T cell responses. Vaccine 2014, 32, 1174.
Englund, E. A.; Wang, D. Y.; Fujigaki, H.; Sakai, H.; Micklitsch, C. M.; Ghirlando, R.; Martin-Manso, G.; Pendrak, M. L.; Roberts, D. D.; Durell, S. R.; Appella, D. H. Programmable multivalent display of receptor ligands using peptide nucleic acid nanoscaffolds. Nat Commun 2012, 3.
Huang, Z. H.; Shi, L.; Ma, J. W.; Sun, Z. Y.; Cai, H.; Chen, Y. X.; Zhao, Y. F.; Li, Y. M. a Totally Synthetic, Self-Assembling, Adjuvant-Free MUC1 Glycopeptide Vaccine for Cancer Therapy. J Am Chem Soc 2012, 134, 8730.
Ino, A.; Dickerson, T. J.; Janda, K. D. Positional linker effects in haptens for cocaine immunopharmacotherapy. Bioorg Med Chem Lett 2007, 17, 4280.
Kosten, T.; Domingo, C.; Orson, F.; Kinsey, B. Vaccines against stimulants: cocaine and MA. Brit J Clin Pharmaco 2014, 77, 368.
Kosten, T. R.; Domingo, C. B.; Shorter, D.; Orson, F.; Green, C.; Somoza, E.; Sekerka, R.; Levin, F. R.; Mariani, J. J.; Stitzer, M.; Tompkins, D. A.; Rotrosen, J.; Thakkar, V.; Smoak, B.; Kampman, K. Vaccine for cocaine dependence: A randomized double-blind placebo-controlled efficacy trial. Drug and alcohol dependence 2014, 140, 42.
Kushner, A. M.; Guan, Z. B. Modular Design in Natural and Biomimetic Soft Materials. Angew Chem Int Edit 2011, 50, 9026.
Matson, J. B.; Stupp, S. I. Self-assembling peptide scaffolds for regenerative medicine. Chem Commun 2012, 48, 26.
Moreno, A. Y.; Janda, K. D. Immunopharmacotherapy: vaccination strategies as a treatment for drug abuse and dependence. Pharmacol. Biochem. Behav. 2009, 92, 199.
Moreno, A. Y.; Janda, K. D. Current challenges for the creation of effective vaccines against drugs of abuse. Expert. Rev. Vaccines. 2011, 10, 1637.
Orson, F. M.; Rossen, Shen, X.; Lopez, A. Y.; Wu, Y', Kosten, T. R. Spontaneous developmnent of IgM anti-cocaine antibodies in habitual cocaine users: effect on IgG antibody responses to a cocaine cholera toxin B conjugate vaccine. Am J. Addict. 2013, 22, 169.
Orson, F. M.; Wang, R. F.; Brimijoin, S.; Kinsey, B. M.; Singh, R. A. K.; Ramakrishnan, M.; Wang, H. Y.; Kosten, T. R. The future potential for cocaine vaccines. Expert Opin Biol Th 2014, 14, 1271.
Pellegrino, T. C.; Dunn, K. L.; Bayer, B. M. Mechanisms of cocaine-induced decreases in immune cell function. Int Immunopharmacol. 2001, 1, 665.
Petkau-Milroy, K.; Brunsveld, L. Supramolecular chemical biology; bioactive synthetic self-assemblies. Org Biomol Chem 2013, 11, 219.
Ramakrishnan, M.; Kinsey, B. M.; Singh, R. A.; Kosten, T. R.; Orson, F. M. Hapten Optimization for Cocaine Vaccine with Improved Cocaine Recognition. Chem Biol Drug Des 2014, 84, 354.
Rudra, J. S.; Tian, Y. F.; Jung, J. P.; Collier, J. H. A self-assembling peptide acting as an immune adjuvant. P Natl Acad Sci USA 2010, 107, 622.
Rudra, J. S.; Mishra, S.; Chong, A. S.; Mitchell, R. A.; Nardin, E. H.; Nussenzweig, V.; Collier, J. H. Self-assembled peptide nanofibers raising durable antibody responses against a malaria epitope. Biomaterials 2012, 33, 6476.
Rudra, J. S.; Sun, T.; Bird, K. C.; Daniels, M. D.; Gasiorowski, J. Z.; Chong, A. S.; Collier, J. H. Modulating Adaptive Immune Responses to Peptide Self-Assemblies. Acs Nano 2012, 6, 1557.
Shen, X. Y.; Orson, F. M.; Kosten, T. R. Vaccines against drug abuse. Clin. Pharmacol. Ther. 2012, 91, 60.
Smith, D. M.; Simon, J. K.; Baker, J. R. Applications of nanotechnology for immunology. Nat Rev Immunol 2013, 13, 592.
Valencia, F.; Bubar, M. J.; Milligan, G.; Cunningham, K. A.; Bourne, N. Influence of methamphetamine on genital herpes simplex virus type 2 infection in a mouse model. Sexually transmitted diseases 2012, 39, 720.
Wen, Y.; Collier, J. H. Supramolecular peptide vaccines: tuning adaptive immunity. Curr Opin Immunol 2015, 35, 73.
Yan, J.; Mei, F. C.; Cheng, H.; Lao, D. H.; Hu, Y.; Wei, J.; Patrikeev, I.; Hao, D.; Stutz, S. J.; Dineley, K. T.; Motamedi, M.; Hommel, J. D.; Cunningham, K. A.; Chen, J.; Cheng, X. Enhanced Leptin Sensitivity, Reduced Adiposity, and Improved Glucose Homeostasis in Mice Lacking Exchange Protein Directly Activated by Cyclic AMP Isoform 1. Molecular and cellular biology 2013, 33, 918.
Zhou, J.; Zhang, A.; Klass, T.; Johnson, K. M.; Wang, C. Z.; Ye, Y. P.; Kozikowski, A. P. Biaryl analogues of conformationally constrained tricyclic tropanes as potent and selective norepinephrine reuptake inhibitors: synthesis and evaluation of their uptake inhibition at monoamine transporter sites. Journal of medicinal chemistry 2003, 46, 1997.

1. A peptide fibril comprising a plurality of self-assembling peptides coupled to an antigen having a chemical formula of Formula I 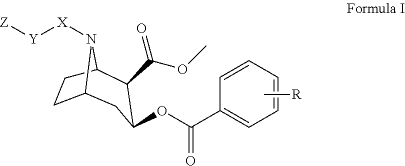
wherein X is —CH₂—, —CO—, or —CS—;
Y is a substituted alkane, alkene, alkyne, or aromatic rings;
Z is —CO₂H—, —OH, N₃, —NH₂, —CHO, —SH, —CH═CH₂, —C≡CH, or 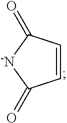
and
R is hydrogen, hydroxyl, nitro, mercapto, cyano, azido, alkyl, heteroalkyl, alkoxy, halogen, amino, oxo, or alkylsulfonyl.

2. The fibril of claim 1, wherein R is H, F, Cl, or Br.

3. The fibril of claim 1, wherein R is located at position 2, 3, 4, 5, or 6.

4. The fibril of claim 1, wherein the self-assembling peptide is selected from:
(SEQ ID NO: 1)
FKFEFKFE;
(SEQ ID NO: 2)
QQKFQFQFEQQ;
(SEQ ID NO: 3)
KFQFQFE;
(SEQ ID NO: 4)
QQRFQFQFEQQ;
(SEQ ID NO: 5)
QQRFQWQFEQQ;
(SEQ ID NO: 6)
FEFEFKFKFEFEFKFK;
(SEQ ID NO: 7)
QQRFEWEFEQQ;
(SEQ ID NO: 8)
QQXFWXFQQQ (where X denotes ornithine);
(SEQ ID NO: 9)
FKFEFKFEFKFE;
(SEQ ID NO: 10)
FKFQFKFQFKFQ;
(SEQ ID NO: 11)
AEAKAEAKAEAKAEAK;
(SEQ ID NO: 12)
AEAEAKAKAEAEAKAK;
(SEQ ID NO: 13)
AEAEAEAEAKAKAKAK;
(SEQ ID NO: 14)
RADARADARADARADA;
(SEQ ID NO: 15)
RARADADARARADADA;
(SEQ ID NO: 16)
SGRGYBLGGQGAGAAAAAGGAGQGGYGGLGSQG;
(SEQ ID NO: 17)
EWEXEXEXEX (Where X = V, A, S, or P);
(SEQ ID NO: 18)
WKXKXKXKXK (Where X = V, A, S, or P);
(SEQ ID NO: 19)
KWKVKVKVKVKVKVK (Where X = V, A, S, or P);
(SEQ ID NO: 20)
LLLLKKKKKKKKLLLL;
(SEQ ID NO: 21)
VKVKVKVKVDPPTKVKVKVKV;
(SEQ ID NO: 22)
VKVKVKVKVDPPTKVKTKVKV;
(SEQ ID NO: 23)
KVKVKVKVKDPPSVKVKVKVK;
(SEQ ID NO: 24)
VKVKVKVKVDPPSKVKVKVKV;
or
(SEQ ID NO: 25)
VKVKVKTKVDPPTKVKTKVKV.

5. The fibril of claim 1, wherein the self-assembling peptide has the amino acid sequence FKFEFKFE (SEQ ID NO:1).

6. The fibril of claim 1, wherein the antigen is covalently coupled to the self-assembling peptides.

7. The fibril of claim 1, wherein the antigens are covalently coupled to a terminus of the self-assembling peptides.

8. The fibril of claim 1, wherein the antigens are covalently coupled to the amino terminus of the self-assembling peptides

9. The fibril of claim 1, wherein the antigens are covalently coupled to the peptide fibril by a peptide linker.

10. The fibril of claim 9, wherein the peptide linker is a poly-glycine linker.

11. The fibril of claim 10, wherein the poly-glycine linker is a triple glycine linker.

12. The fibril of claim 1, wherein the amino acids are L-amino acids, D amino acids, or a combination thereof.

13. A self-assembling antigen composition comprising an antigen having a chemical formula of Formula I 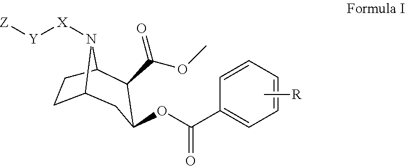
wherein X is —CH₂—, —CO—, or —CS—;
Y is a substituted alkane, alkene, alkyne, or aromatic rings;
Z is —CO₂H—, —OH, N₃, —NH₂, —CHO, —SH, —CH═CH₂, —C≡CH, or 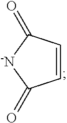
R is hydrogen, hydroxyl, nitro, mercapto, cyano, azido, alkyl, heteroalkyl, alkoxy, halogen, amino, oxo, or alkylsulfonyl; and
coupled to a fibril-forming peptide.

14. The composition of claim 13, wherein the fibril-forming peptide has an amino acid sequence of FKFEFKFE (SEQ ID NO:1)

15. The composition of claim 14, wherein the antigen is coupled to the amino terminus of the fibril forming peptide.

16. The composition of claim 15, wherein the antigen is coupled to the fibril forming peptide by a triple glycine linker.

17. A method of inducing an immune response comprising administering the fibril of claim 1 in an amount sufficient to induce an immune response.

18. A method of treating a subject having cocaine use condition comprising administering to the subject an effective amount of the fibril of claim 1.


SEQUENCE LISTING (28 sequences)

SEQ ID NO: 1 - protein, 8 aa, Artificial Sequence
  Synthetic Peptide
FKFEFKFE

SEQ ID NO: 2 - protein, 11 aa, Artificial Sequence
  Synthetic Peptide
QQKFQFQFEQQ

SEQ ID NO: 3 - protein, 7 aa, Artificial Sequence
  Synthetic Peptide
KFQFQFE

SEQ ID NO: 4 - protein, 11 aa, Artificial Sequence
  Synthetic Peptide
QQRFQFQFEQQ

SEQ ID NO: 5 - protein, 11 aa, Artificial Sequence
  Synthetic Peptide
QQRFQWQFEQQ

SEQ ID NO: 6 - protein, 16 aa, Artificial Sequence
  Synthetic Peptide
FEFEFKFKFEFEFKFK

SEQ ID NO: 7 - protein, 11 aa, Artificial Sequence
  Synthetic Peptide
QQRFEWEFEQQ

SEQ ID NO: 8 - protein, 11 aa, Artificial Sequence
  Synthetic Peptide
  MISC_FEATURE (3)..(3): X is ornithine
  MISC_FEATURE (5)..(5): X is ornithine
  MISC_FEATURE (7)..(7): X is ornithine
QQXFXWXFQQQ

SEQ ID NO: 9 - protein, 12 aa, Artificial Sequence
  Synthetic Peptide
FKFEFKFEFKFE

SEQ ID NO: 10 - protein, 12 aa, Artificial Sequence
  Synthetic Peptide
FKFQFKFQFKFQ

SEQ ID NO: 11 - protein, 16 aa, Artificial Sequence
  Synthetic Peptide
AEAKAEAKAEAKAEAK

SEQ ID NO: 12 - protein, 16 aa, Artificial Sequence
  Synthetic Peptide
AEAEAKAKAEAEAKAK

SEQ ID NO: 13 - protein, 16 aa, Artificial Sequence
  Synthetic Peptide
AEAEAEAEAKAKAKAK

SEQ ID NO: 14 - protein, 16 aa, Artificial Sequence
  Synthetic Peptide
RADARADARADARADA

SEQ ID NO: 15 - protein, 16 aa, Artificial Sequence
  Synthetic Peptide
RARADADARARADADA

SEQ ID NO: 16 - protein, 33 aa, Artificial Sequence
  Synthetic Peptide
SGRGYBLGGQGAGAAAAAGGAGQGGYGGLGSQG

SEQ ID NO: 17 - protein, 10 aa, Artificial Sequence
  Synthetic Peptide
  MISC_FEATURE (4)..(4): X is V, A, S, or P
  MISC_FEATURE (6)..(6): X is V, A, S, or P
  MISC_FEATURE (8)..(8): X is V, A, S, or P
  MISC_FEATURE (10)..(10): X is V, A, S, or P
EWEXEXEXEX

SEQ ID NO: 18 - protein, 10 aa, Artificial Sequence
  Synthetic Peptide
  MISC_FEATURE (3)..(3): X is V, A, S, or P
  MISC_FEATURE (5)..(5): X is V, A, S, or P
  MISC_FEATURE (7)..(7): X is V, A, S, or P
  MISC_FEATURE (9)..(9): X is V, A, S, or P
WKXKXKXKXK

SEQ ID NO: 19 - protein, 15 aa, Artificial Sequence
  Synthetic Peptide
KWKVKVKVKVKVKVK

SEQ ID NO: 20 - protein, 16 aa, Artificial Sequence
  Synthetic Peptide
LLLLKKKKKKKKLLLL

SEQ ID NO: 21 - protein, 21 aa, Artificial Sequence
  Synthetic Peptide
VKVKVKVKVDPPTKVKVKVKV

SEQ ID NO: 22 - protein, 21 aa, Artificial Sequence
  Synthetic Peptide
VKVKVKVKVDPPTKVKTKVKV

SEQ ID NO: 23 - protein, 21 aa, Artificial Sequence
  Synthetic Peptide
KVKVKVKVKDPPSVKVKVKVK

SEQ ID NO: 24 - protein, 21 aa, Artificial Sequence
  Synthetic Peptide
VKVKVKVKVDPPSKVKVKVKV

SEQ ID NO: 25 - protein, 21 aa, Artificial Sequence
  Synthetic Peptide
VKVKVKTKVDPPTKVKTKVKV

SEQ ID NO: 26 - protein, 4 aa, Artificial Sequence
  Synthetic Peptide
SGSG

SEQ ID NO: 27 - protein, 5 aa, Artificial Sequence
  Synthetic Peptide
GGAAY

SEQ ID NO: 28 - protein, 11 aa, Artificial Sequence
  Synthetic Peptide
GGGFKFEFKFE